(19) United States
(12) Patent Application Publication
(10) Pub. No.: US 2006/0100259 A1
(43) Pub. Date: May 11, 2006

(54) Process for the preparation of substituted triazole compounds

(76) Inventors: David C. Palmer, Doylestown, PA (US); Kirk L. Sorgi, Doylestown, PA (US); Tong Xiao, Princeton, NJ (US); Sergio Cesco-Cancian, Bethlehem, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/040,757

(22) Filed: Jan. 21, 2005

Related U.S. Application Data
(60) Provisional application No. 60/543,721, filed on Feb. 11, 2004.
(60) Provisional application No. 60/623,681, filed on Oct. 29, 2004.

(51) Int. Cl. A61K 31/4196; C07D 249/14; C07D 249/08

(52) U.S. Cl. 514/383; 548/264.8

(57) ABSTRACT

The present invention is directed to a novel process for the preparation of substituted triazole compounds, useful in the treating or ameliorating a selective kinase or dual-kinase mediated disorder. The process of the present invention preferentially produces the desired regioisomer of the substituted triazole compounds.

### CROSS REFERENCE TO RELATED APPLICATIONS

This application claims the benefit of U. S. Provisional Application 60/543,721, filed on Feb. 11, 2004 and U.S. Provisional Application 60/623,681, filed on Oct. 29, 2004, which are incorporated by reference herein in their entirety.

### FIELD OF THE INVENTION

The present invention is directed to a novel process for the preparation of substituted triazole compounds, useful in the treating or ameliorating a selective kinase or dual-kinase mediated disorder. The process of the present invention preferentially produces the regioisomer of compounds of formula (I).

### BACKGROUND OF THE INVENTION

The present invention is directed to a process for the preparation of compounds of formula (I). Compounds of formula (I) are selective kinase or dual-kinase inhibitors useful in a method for treating or ameliorating a kinase or dual-kinase mediated disorder. In particular, the kinase is selected from a cyclin dependent kinase and a tyrosine kinase. More particularly, the kinase is selected from cyclin dependent kinase-1, cyclin-dependent kinase-2, cyclin-dependent kinase-4, vascular endothelial growth factor receptor-2, endothelial growth factor receptor or human epidermal growth factor receptor-2.

Lin et al., in PCT publication WO02/057240, which is herein incorporated by reference, disclose several processes for the preparation of substituted triazole compounds. The processes disclosed by Lin et al., require the use of hydrazine (which is toxic, mutagenic and potentially explosive) and/or require chromatographic separation of product and/or intermediates, and/or result in the formation of multiple regioisomers (which need to be separated by whatever means), making these processes unsuitable for large scale production.

Known processes for the preparation of substituted triazoles which comprise reacting unsubstituted triazoles with suitably selected reagents result in the formation of regioisomers of the substituted triazole compounds. This occurs because the reagent(s) reacted with the unsubstituted triazole will react with more than one nitrogen atom of the triazole, thereby resulting in compounds with different substitution patterns—i.e. regioisomers.

Thus there remains a need for a process for the preparation of substituted triazole compounds, wherein the regioisomer of formula (I), as hereinafter defined, is preferentially prepared.

### SUMMARY OF THE INVENTION

The present invention provides a compound of Formula (I): 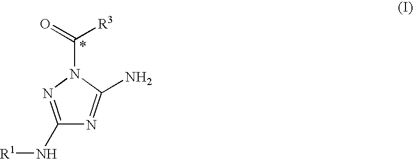
wherein
R₁ is selected from the group consisting of C₁₋₈alkyl, cycloalkyl, heterocyclyl, aryl and heteroaryl
   wherein the cycloalkyl, heterocyclyl, aryl and heteroaryl are substituted with a substituent selected from the group consisting of:
   (a) C₁₋₈alkyl (optionally substituted on a terminal carbon with a substituent selected from the group consisting of —C(O)H, —C(O)(C₁₋₈)alkyl, —CO₂(C₁₋₈)alkyl, amino, C₁₋₈alkylamino, di(C₁₋₈alkyl)amino, cyano, (halo)₁₋₃, hydroxy, nitro, cycloalkyl, heterocyclyl, aryl and heteroaryl),
   (b) C₁₋₈alkoxy (optionally substituted on a terminal carbon with a substituent selected from the group consisting of (halo)₁₋₃ and hydroxy),
   (c) —C(O)H, —C(O)(C₁₋₈)alkyl;
(d) —CO₂(C₁₋₈)alkyl;
   (e) amino (substituted with two substituents independently selected from the group consisting of hydrogen, C₁₋₈alkyl and —SO₂—(C₁₋₈)alkyl),
   (f) —C(O)amino (wherein amino is substituted with two substituents independently selected from the group consisting of hydrogen and C₁₋₈alkyl),
   (g) —SO₂— {substituted with one substituent selected from the group consisting of heterocyclyl and amino (wherein amino is substituted with two substituents independently selected from the group consisting of hydrogen, C₁₋₈alkyl, —C₁₋₈alkylamino (wherein amino is substituted with two substituents independently selected from the group consisting of hydrogen and C₁₋₈alkyl) and heteroaryl)},
   (h) cycloalkyl, heterocyclyl, aryl and heteroaryl
      (wherein the cycloalkyl, heterocyclyl, aryl and heteroaryl are optionally substituted with 1 to 3 substituents independently selected from the group consisting of cyano, halo, hydroxy and nitro;
      and wherein the heterocyclyl is optionally substituted with 1 to 2 oxo substituents; and, wherein cycloalkyl, heterocyclyl, aryl and heteroaryl are optionally substituted with a substituent selected from the group consisting of C₁₋₈alkyl (wherein alkyl is optionally substituted on a terminal carbon with a substituent selected from the group consisting of amino, C₁₋₈alkylamino, di(C₁₋₈alkyl)amino, cyano, (halo)₁₋₃, hydroxy and nitro), C₁₋₈alkoxy, amino, C₁₋₈alkylamino and di(C₁₋₈alkyl)amino);
R₃ is selected from the group consisting of: C₁₋₈alkyl, C₂₋₈alkenyl, C₂₋₈alkynyl (wherein the C₁₋₈alkyl, C₂₋₈alkenyl and C₂₋₈alkynyl are optionally substituted on a terminal carbon with a substituent selected from the group consisting of —C(O)H, —C(O)(C₁₋₈)alkyl, —CO₂(C₁₋₈)alkyl, amino, C₁₋₈alkylamino, di(C₁₋₈alkyl)amino, cyano, (halo)₂₋₃, hydroxy, nitro, aryl and heteroaryl (wherein aryl and heteroaryl are optionally substituted with 1 to 5 substituents independently selected from the group consisting of C₁₋₈alkyl, cyano, (halo)₁₋₃(C₁₋₈)alkyl, (halo)₁₋₃(C₁₋₈)alkoxy, hydroxy, hydroxy(C₁₋₈)alkyl, hydroxy(C₁₋₈)alkoxy and nitro)},
cycloalkyl, heterocyclyl, aryl, heteroaryl
   (wherein the cycloalkyl, heterocyclyl, aryl and heteroaryl are optionally substituted with 1 to 3 substituents independently selected from the group consisting of cyano, hydroxy and nitro;
   wherein the aryl and heteroaryl are optionally substituted with (halo)₁₋₃;
   and wherein the cycloalkyl, heterocyclyl, aryl and heteroaryl are optionally substituted with 1 to 2 substituents independently selected from the group consisting of:
   (a) C₁₋₈alkyl, C₂₋₈alkenyl (wherein the C₁₋₈alkyl and C₂₋₈alkenyl are optionally substituted on a terminal carbon with a substituent selected from the group consisting of —C(O)H, —C(O)(C₁₋₈)alkyl, —CO₂(C₁₋₈)alkyl, amino, C₁₋₈alkylamino, di(C₁₋₈alkyl)amino, cyano, (halo)₂₋₃, hydroxy, nitro, cycloalkyl, heterocyclyl, aryl and heteroaryl),
   (b) —CH(OH)—(C₁₋₈)alkyl,
   (c) C₁₋₈alkoxy (optionally substituted on a terminal carbon with a substituent selected from the group consisting of (halo)₂₋₃ and hydroxy),
   (d) —C(O)H, —C(O)(C₁₋₈)alkyl;
   (e) —CO₂(C₁₋₈)alkyl;
   (f) amino (substituted with two substituents independently selected from the group consisting of hydrogen, C₁₋₈alkyl and —C(O)(C₁₋₈)alkyl),
   (g) —C(O)amino (wherein amino is substituted with two substituents independently selected from the group consisting of hydrogen and C₁₋₈alkyl),
   (h) —SO₂— {substituted with one substituent selected from the group consisting of heterocyclyl and amino (wherein amino is substituted with two substituents independently selected from the group consisting of hydrogen, C₁₋₈alkyl and —C₁₋₈alkylamino (wherein amino is substituted with two substituents independently selected from the group consisting of hydrogen and C₁₋₈alkyl))},
   (i) —NH—SO₂—(C₁₋₈)alkyl,
   (j) cycloalkyl, heterocyclyl (optionally substituted with 1 to 2 oxo substituents), aryl and heteroaryl} and amino;
wherein the amino group is substituted with two substituents independently selected from the group consisting of hydrogen, C₁₋₈alkyl, cycloalkyl, aryl and heteroaryl (wherein the cycloalkyl, aryl and heteroaryl are optionally substituted with 1 to 5 substituents independently selected from the group consisting of C₁₋₈alkyl, cyano, (halo)₁₋₃(C₁₋₈)alkyl, (halo)₁₋₃(C₁₋₈)alkoxy, hydroxy, hydroxy(C₁₋₈)alkyl, hydroxy(C₁₋₈)alkoxy and nitro);

provided that when R³ is aryl or heteroaryl, wherein the aryl or heteroaryl is optionally substituted with a —(CH₂)₀₋₂—CO₂(C₁₋₈)alkyl group, then the —(CH₂)_{0-2—CO}₂(C₁₋₈)alkyl group is not bound at the ortho position relative to the bond identified by the asterisk in the compound of formula (I);

provided further that when R³ is cycloalkyl or a heterocyclyl, wherein the cycloalkyl or heterocyclyl is optionally substituted, then the substituent on the cycloalkyl or heterocyclyl is other than —(CH₂)₀₋₂—CO₂(C₁₋₈)alkyl;

and pharmaceutically acceptable salts thereof;

comprising

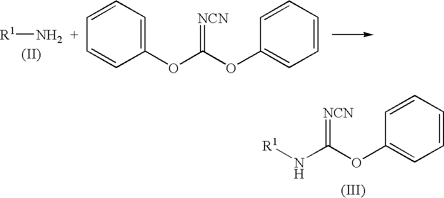

reacting a suitably substituted compound of formula (II) with diphenyl cyanocarbonimidate, in a first organic solvent, to yield the corresponding compound of formula (III);

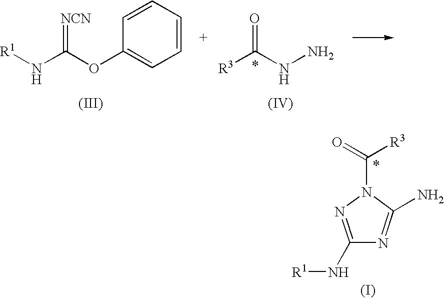

reacting the compound of formula (III) with a suitably substituted compound of formula (IV), in a second organic solvent, to yield the corresponding compound of formula (I).

The present invention is further directed to a process for the preparation of a compound of formula (Ia)

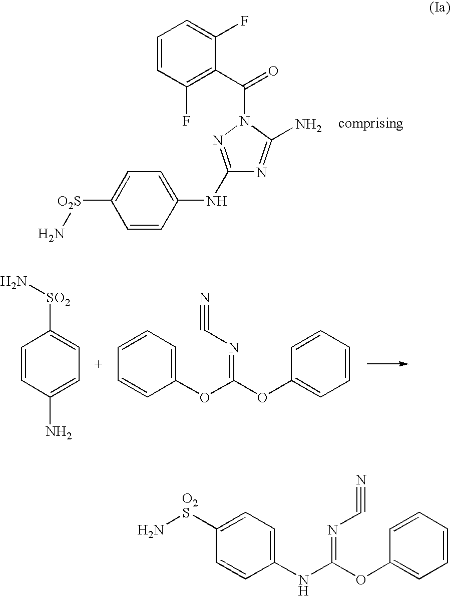

reacting 4-aminobenzenesulfonamide with diphenyl cyanocarbonimidate, in a first organic solvent, to yield N-[4-(aminosulfonyl)phenyl]-N′-cyanocarbamidic acid phenyl ester;

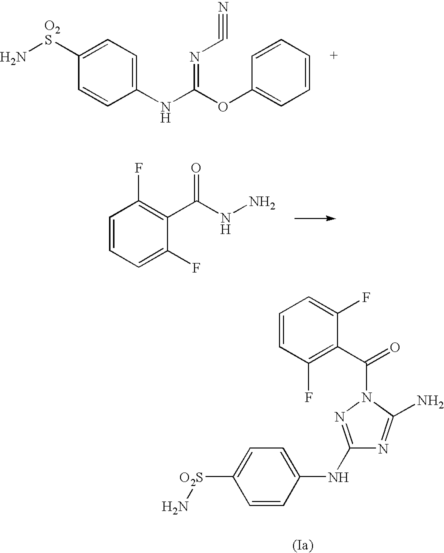

reacting N-[4-(aminosulfonyl)phenyl]-N′-cyanocarbamidic acid phenyl ester with 2,6-difluorobenzoic acid hydrazide, in a second organic solvent, to yield the corresponding compound of formula (Ia).

The present invention is further directed to novel crystalline forms of the compound of formula (Ia) and to novel processes for the preparation of said crystalline forms of the compound of formula (Ia).

The present invention is further directed to novel crystalline salts of the compound of formula (Ia). More particularly, the present invention is directed to CH₃SO₃H, HCl, HBr and H₂SO₄ salts of the compound of formula (Ia). The present invention is further directed to novel processes for the preparation of said salts of the compound of formula (Ia). The present invention is further directed to pharmaceutical composition comprising any of the salts described herein and a pharmaceutically acceptable carrier.

The present invention is further directed to a product prepared according to any of the processes disclosed herein.

Illustrative of the invention is a pharmaceutical composition comprising a pharmaceutically acceptable carrier and a compound prepared according to any of the processes described herein. An illustration of the invention is a pharmaceutical composition made by mixing a compound prepared according to any of the processes described herein and a pharmaceutically acceptable carrier. Illustrating the invention is a process for making a pharmaceutical composition comprising mixing a compound prepared according to any of the processes described herein and a pharmaceutically acceptable carrier.

### DETAILED DESCRIPTION OF THE INVENTION

The present invention is directed to a process for the preparation of compounds of formula (I)

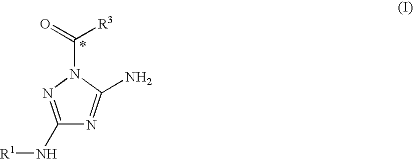

wherein R¹ and R³ are as defined above. Compounds of formula (I) are useful in treating or ameliorating a selective kinase or dual-kinase mediated disorder.

In an embodiment of the present invention is a process for the preparation of a compound of formula (Ia).

In an embodiment of the present invention R¹ is selected from the group consisting of aryl and heteroaryl, wherein the aryl or heteroaryl group is optionally substituted as defined above. Preferably, R¹ is aryl, wherein the aryl group is optionally substituted with aminosulfonyl. More preferably, R¹ is 4-aminosulfonylphenyl.

In an embodiment of the present invention R³ is selected from the group consisting of aryl and heteroaryl, wherein the aryl or heteroaryl group is optionally substituted as defined above. Preferably, R³ is aryl, wherein the aryl is substituted with 1 to 3 halo. More preferably, R³ is 2,6-difluorophenyl.

In an embodiment of the present invention R¹ is 4-aminosulfonylphenyl and R³ is 2,6-difluorophenyl.

In an embodiment of the present invention, the process of the present invention, prepares the regioisomer of formula (I) in a ratio of greater than or equal to 10:1, preferably at a ratio of greater than or equal to 25:1, more preferably, at a ratio of greater than or equal to 50:1.

In an embodiment of the present invention, the process of the present invention, prepares the regioisomer of formula (Ia) in a ratio of greater than or equal to 10:1, preferably at a ratio of greater than or equal to 25:1, more preferably, at a ratio of greater than or equal to 50:1.

Unless specified otherwise, the term “alkyl” refers to a saturated straight or branched chain consisting solely of 1-8 hydrogen substituted carbon atoms; preferably, 1-6 hydrogen substituted carbon atoms; and, most preferably, 1-4 hydrogen substituted carbon atoms. The term “alkenyl” refers to a partially unsaturated straight or branched alkyl chain that contains at least one double bond. The term “alkynyl” refers to a partially unsaturated straight or branched alkyl chain that contains at least one triple bond. The term “alkoxy” refers to—O-alkyl, where alkyl is as defined supra.

The term “cycloalkyl” refers to a saturated or partially unsaturated cyclic alkyl ring consisting of 3-8 hydrogen substituted carbon atoms. Examples include, and are not limited to, cyclopropyl, cyclopentyl, cyclohexyl or cycloheptyl.

The term “heterocyclyl” refers to a saturated or partially unsaturated ring having five members of which at least one member is a N, O or S atom and which optionally contains one additional O atom or one, two or three additional N atoms; a saturated or partially unsaturated ring having six members of which one, two or three members are a N atom; a saturated or partially unsaturated bicyclic ring having nine members of which at least one member is a N, O or S atom and which optionally contains one, two or three additional N atoms; and, a saturated or partially unsaturated bicyclic ring having ten members of which one, two or three members are a N atom. Examples include, and are not limited to, pyrrolinyl, pyrrolidinyl, dioxolanyl, imidazolinyl, imidazolidinyl, pyrazolinyl, pyrazolidinyl, piperidinyl, morpholinyl or piperazinyl.

The term “aryl” refers to an aromatic monocyclic ring system containing 6 hydrogen substituted carbon atoms, an aromatic bicyclic ring system containing 10 hydrogen substituted carbon atoms or an aromatic tricyclic ring system containing 14 hydrogen substituted carbon atoms. Examples include, and are not limited to, phenyl, naphthalenyl or anthracenyl.

The term “heteroaryl” refers to an aromatic monocyclic ring system containing five members of which at least one member is a N, O or S atom and which optionally contains one, two or three additional N atoms, an aromatic monocyclic ring having six members of which one, two or three members are a N atom, an aromatic bicyclic ring having nine members of which at least one member is a N, O or S atom and which optionally contains one, two or three additional N atoms and an aromatic bicyclic ring having ten members of which one, two or three members are a N atom. Examples include, and are not limited to, furyl, thienyl, pyrrolyl, oxazolyl, thiazolyl, imidazolyl, pyrazolyl, isoxazolyl, isothiazolyl, pyridinyl, pyridazinyl, pyrimidinyl, pyrazinyl, indolyl, indazolyl, quinolinyl or isoquinolinyl.

The term “halo” or “halogen” refers to a fluoro, chloro, bromo or iodo atom.

When a particular group is “substituted” (e.g., Ph, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

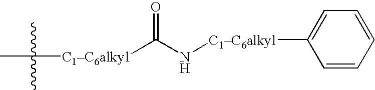

Abbreviations used in the specification, particularly the Schemes and Examples, are as follows:

DIPEA or DIEA=Diisopropylethylamine

DMA=Dimethyl Acetamide

DME=1,2-Dimethoxyethane

DMF=N,N-Dimethylformamide

DMSO=Dimethylsulfoxide

DPCCI Diphenylcyanocarbonimidate

HPLC=High Pressure Liquid Chromatography

IPA=Isopropyl Alcohol

MeCN=Acetonitrile

MeOH=Methanol

MTBE=Methyl-t-butyl ether

NMP=N-Methyl pyrrolidone

Ph=Phenyl

Pyr=Pyridine

TEA=Triethylamine

THF=Tetrahydrofuran

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The present invention relates to a process for preparing a compounds of formula (I) as more fully described in the schemes below.

Compounds of formula (I) may be prepared according to the process outlined in Scheme 1.

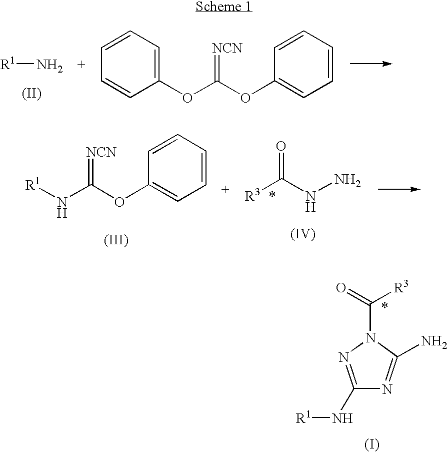

Accordingly, a suitably substituted compound of formula (II), a known compound or compound prepared by known methods, is reacted with diphenyl cyanocarbonimidate, a known compound;

optionally in the presence of a Lewis acid catalyst such as ZnCl₂, TiCl₄, SnCl₄, BF₃.Etherate, and the like, or a first inorganic or organic base such as Na₂CO₃, K₂CO₃, NaHCO₃, Cs₂CO₃, NaOH, KOH, TEA, DIPEA, NaO(C₁₋₄alkyl) (for example NaOCH₂CH₃, NaOCH₃, NaOC(CH₃)₃, and the like), KO(C₁₋₄alkyl) (for example KO-tert-butyl, and the like), pyridine, and the like, more preferably a first organic base, more preferably still a tertiary amine base such as TEA, DIPEA, pyridine, and the like, more preferably still pyridine;

in a first organic solvent such as methanol, ethanol, IPA, n-butanol, tert-butanol, acetonitrile, pyridine, THF, IPA, DMF, DME, DMA, sulfolane, and the like, preferably in pyridine;

preferably, at a temperature in the range of from about room temperature to about 120° C.;

more preferably, in pyridine, at about room temperature;

to yield the corresponding compound of formula (III).

The compound of formula (III) is reacted with a suitably substituted compound of formula (IV), a known compound or compound prepared by known methods;

preferably in the presence of a second organic or inorganic base, Na₂CO₃, K₂CO₃, NaHCO₃, Cs₂CO₃, NaOH, KOH, TEA, DIPEA, NaO(C₁₋₄alkyl) (for example NaOCH₂CH₃, NaOCH₃, NaOC(CH₃)₃, and the like), KO(C₁₋₄alkyl) (for example KO-tert-butyl, and the like), pyridine, and the like, more preferably a second organic base, more preferably still a tertiary amine base such as TEA, DIPEA, pyridine, and the like, more preferably still pyridine;

in a second organic solvent such as methanol, ethanol, IPA, n-butanol, tert-butanol, acetonitrile, pyridine, THF, IPA, DMF, DME, DMA, sulfolane, and the like, preferably in pyridine;

preferably at a temperature in the range of from about room temperature to about 120° C.;

more preferably, in pyridine, at a temperature in the range of from about 80 to about 90° C.;

to yield the corresponding compound of formula (I).

The present invention is further directed to a process for the preparation of a compound of formula (Ia)

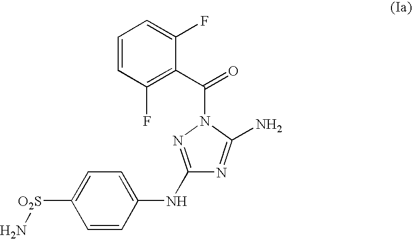

as outlined in Scheme 2 below.

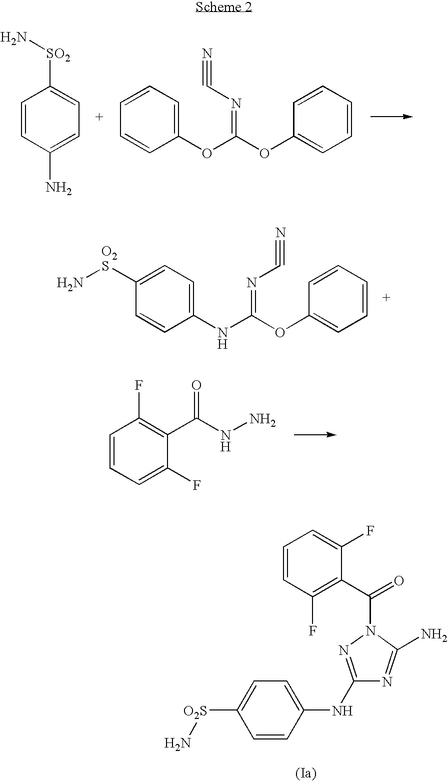

Accordingly, 4-aminobenzenesulfonamide, a known compound, is reacted with diphenyl cyanocarbonimidate, a known compound;

optionally in the presence of a Lewis acid catalyst such as ZnCl₂, TiCl₄, SnCl₄, BF₃.Etherate, and the like, or a first inorganic or organic base such as Na₂CO₃, K₂CO₃, NaHCO₃, Cs₂CO₃, NaOH, KOH, TEA, DIPEA, NaO(C₁₋₄alkyl) (for example NaOCH₂CH₃, NaOCH₃, NaOC(CH₃)₃, and the like), KO(C₁₋₄alkyl) (for example KO-tert-butyl, and the like), pyridine, and the like, more preferably a first organic base, more preferably still a tertiary amine base such as TEA, DIPEA, pyridine, and the like, more preferably still pyridine;

in a first organic solvent such as methanol, ethanol, IPA, n-butanol, tert-butanol, acetonitrile, pyridine, THF, IPA, DMF, DME, DMA, sulfolane, and the like, preferably in pyridine;

preferably at a temperature in the range of from about room temperature to about 120° C.;

more preferably, in pyridine, at about room temperature;

to yield the corresponding N-[4-(aminosulfonyl)phenyl]-N′-cyanocarbamidic acid phenyl ester.

The N-[4-(aminosulfonyl)phenyl]-N′-cyanocarbamidic acid phenyl ester is reacted with a 2,6-difluorobenzoic acid hydrazide, a known compound;

preferably in the presence of a second organic or inorganic base, Na₂CO₃, K₂CO₃, NaHCO₃, Cs₂CO₃, NaOH, KOH, TEA, DIPEA, NaO(C₁₋₄alkyl) (for example NaOCH₂CH₃, NaOCH₃, NaOC(CH₃)₃, and the like), KO(C₁₋₄alkyl) (for example KO-tert-butyl, and the like), pyridine, and the like, more preferably a second organic base, more preferably still a tertiary amine base such as TEA, DIPEA, pyridine, and the like, more preferably still pyridine;

in a second organic solvent such as methanol, ethanol, IPA, n-butanol, tert-butanol, acetonitrile, pyridine, THF, IPA, DMF, DME, DMA, sulfolane, and the like, preferably in pyridine;

preferably at a temperature in the range of from about room temperature to about 120° C.;

more preferably, in pyridine, at a temperature in the range of from about 80 to about 90° C.;

to yield the corresponding compound of formula (Ia).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups (e.g. aldehydes, ketones, and the like) on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The process of the present invention was used in the preparation of representative compounds of formula (I) as listed in Tables 1 and 2 below.

TABLE 1

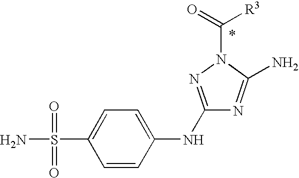

| ID No. | R³ | Calc MW | Meas MW |
|---|---|---|---|
| 1 | phenyl | 358.38 | 359 |
| 2 | 2-chlorophenyl | 392.83 | 393 |
| 3 | 2-methoxyphenyl | 388.41 | 389 |
| 4 | 2-bromophenyl | 437.28 | 438 |
| 5 | 3-nitrophenyl | 403.38 | 404 |
| 6 | 3-trifluoromethylphenyl | 426.38 | 427 |
| 7 | 3-bromophenyl | 437.28 | 438 |
| 8 | 4-methylphenyl | 372.41 | 373 |
| 9 | 4-nitrophenyl | 403.38 | 404 |
| 10 | 4-hydroxyphenyl | 374.38 | 375 |
| 11 | 4-biphenyl | 434.48 | 435 |
| 12 | 4-methoxyphenyl | 388.41 | 389 |
| 13 | 4-trifluoromethylphenyl | 426.38 | 427 |
| 16 | methyl | 296.31 | 297 |
| 17 | 2-furyl | 348.34 | 349 |
| 18 | 2-thienyl | 364.41 | 365 |
| 19 | 3-pyridyl | 359.37 | 360 |
| 28 | 4-chlorophenyl | 392.83 | 393 |

TABLE 2

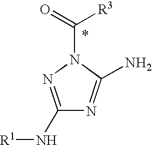

| | | | | | Exact | Exact |
|---|---|---|---|---|---|---|
| ID | | | Calc | Meas | Mass | Mass |
| No. | R³ | R¹ | MW | MW | Calc. | Meas. |
| 24 | phenyl | phenyl | 279.30 | 380 | | |
| 25 | 4-methyl- | phenyl | 293.31 | 294.1 | | |
| | phenyl | | | | | |
| 26 | 4-nitro- | phenyl | 324.29 | 325 | | |
| | phenyl | | | | | |
| 27 | 4-chloro- | phenyl | 313.74 | 314 | | |
| | phenyl | | | | | |
| 29 | 2- | phenyl | 309.32 | 310 | 310.1299 | 310.1306 |
| | methoxy- | | | | | |
| | phenyl | | | | | |
| 30 | 3- | phenyl | 309.32 | 310 | 310.1299 | 310.1302 |
| | methoxy- | | | | | |
| | phenyl | | | | | |
| 31 | 2-furyl | phenyl | 269.26 | 270 | | |
| 32 | 2-furyl | 4-methoxy- | 299.28 | 300.1 | | |
| | | phenyl | | | | |
| 33 | 2-thienyl | 4-methoxy- | 315.36 | 316 | | |
| | | phenyl | | | | |
| 34 | 2-chloro- | 4-methoxy- | 343.77 | 344 | | |
| | phenyl | phenyl | | | | |
| 36 | 3-pyridyl | 3-(6- | 311.30 | 312 | 312.1204 | 312.1209 |
| | | methoxy- | | | | |
| | | pyridyl) | | | | |
| 37 | 2-chloro- | 3-(6- | 344.76 | 345.1 | 345.0681 | 345.0858 |
| | phenyl | methoxy- | | | | |
| | | pyridyl) | | | | |
| 38 | 4-nitro- | 2-(4-methyl- | 345.34 | 346 | | |
| | phenyl | thiazolyl) | | | | |

The present invention is further directed to novel crystalline forms of the compound of formula (Ia). More specifically, the present invention is directed to two novel crystalline forms of the compound of formula (Ia), hereinafter referred to as Forms (Ia-1) and (Ia-2).

The present invention is further directed to novel salt forms of the compound of formula (Ia). In an embodiment, the present invention is directed to novel crystalline salts of the compound of formula (Ia). More specifically, the novel crystalline salts of the compound of formula (Ia) are CH₂SO₃H, HCl, HBr and H₂SO₄ salts of the compound of formula (Ia).

The crystalline forms of the compound of formula (Ia) and the crystalline salts of the compound of formula (Ia) may be characterized by their respective powder X-ray diffraction patterns. Unless otherwise noted, the powder X-ray diffraction patterns were measured using a Phillips X'PERT PRO MPD Diffractometer. The samples were back-loaded into a conventional X-ray holder. Using the X-Celerator detector, the samples were scanned from 3 to 35°2θ at a step size of 0.0170°2θ and a time per step of 10.16 seconds. The effective scan speed was 0.2067°/s. Instrument voltage and current settings of 45 kV and 40 mA were employed. Instrument tolerance 2Θ (2 theta) was 0.03°2Θ. Peaks of relative intensity <5% were not tabulated.

In an embodiment of the present invention is a novel crystalline form of the compound of formula (Ia) hereinafter referred to as Form (Ia-1). Novel crystalline Form (Ia-1) may be prepared according to the process outlined in Scheme 2 above, preferably in the absence of a catalyst and provided that the N-[4-(aminosulfonyl)phenyl]-N′-cyanocarbamidic acid ester is not isolated.

Alternatively, crystalline Forma (Ia-1) may be prepared according to the process outlined in Scheme 2, wherein the N-[4-(aminosulfonyl)phenyl]-N′-cyanocarbamidic acid ester is isolated and then reacted to yield the compound of formula (Ia) as a mixture of Form (Ia-1) and Forma (Ia-2). The mixture of Forma (Ia-1) and Form (Ia-2) is dissolved in an organic solvent such as THF, and the like, then reacted with hydrochloric acid, preferably with concentrated hydrochloric acid, in an amount equal to about one equivalent, to yield the compound of formula (Ia) as its corresponding HCl salt, which is isolated. The isolated HCl salt of the compound of formula (Ia) is suspended in water. The suspension is stirred to a constant pH. Upon dissolution in water, the compound of formula (Ia) precipitates as Form (Ia-1).

Novel crystalline Form (Ia-1) may be characterized by its XRD peaks as listed in Table XRD-1, below. The XRD-spectrum for novel crystalline Form (Ia-1) was manually analyzed to instrument tolerance of 0.03 degrees 2 theta.

TABLE XRD-1
Crystalline Form (Ia-1)

| Pos. [°2Theta] | d-spacing [Å] | Rel. Int. [%] |
|---|---|---|
| 5.21 | 16.95 | 21.24 |
| 10.39 | 8.51 | 14.40 |
| 11.56 | 7.66 | 5.24 |
| 13.71 | 6.46 | 29.54 |
| 15.58 | 5.69 | 87.39 |
| 17.00 | 5.22 | 25.38 |
| 17.20 | 5.16 | 27.26 |
| 18.02 | 4.92 | 40.96 |
| 18.71 | 4.74 | 23.97 |
| 19.24 | 4.61 | 39.50 |
| 19.63 | 4.52 | 54.58 |
| 20.11 | 4.42 | 38.33 |
| 21.27 | 4.18 | 45.19 |
| 21.43 | 4.15 | 47.58 |
| 22.69 | 3.92 | 15.18 |
| 23.20 | 3.83 | 91.38 |
| 23.82 | 3.74 | 100.00 |
| 24.91 | 3.57 | 13.59 |
| 25.55 | 3.49 | 6.03 |
| 26.08 | 3.42 | 35.19 |
| 27.56 | 3.24 | 57.62 |
| 27.78 | 3.21 | 55.67 |
| 28.19 | 3.17 | 53.70 |
| 29.55 | 3.02 | 9.29 |
| 30.09 | 2.97 | 14.96 |
| 31.01 | 2.88 | 5.26 |
| 31.46 | 2.84 | 5.81 |
| 32.22 | 2.78 | 11.43 |
| 32.45 | 2.76 | 11.52 |

In another embodiment of the present invention is a novel crystalline form of the compound of formula (Ia) characterized by the major X-ray diffraction peaks having a relative intensity of greater than or equal to about 10%, as listed in Table XRD-1 above. In another embodiment of the present invention is a novel crystalline form of the compound of formula (Ia) characterized by the major X-ray diffraction peaks having a relative intensity of greater than or equal to about 20%, as listed in Table XRD-1 above.

Novel crystalline Form (Ia-1) may alternatively be characterized by its differential scanning calorimetery (DSC) melt endotherm, which exhibits a peak temperature at about 242° C. The DSC melt endotherm was measured on a TA-Instruments Q1000 MTDSC instrument equipped with an RCS cooling unit, placing a 3 mg sample in a standard aluminum TA-Instrument sample pan and scanning at a heating rate of 10° C./min with a 50 mL/min nitrogen purge.

In an embodiment of the present invention is a novel crystalline form of the compound of formula (Ia), hereinafter referred to as Form (Ia-2). Novel crystalline Form (Ia-2) may be prepared according to the process outlined in Scheme 2 wherein the 4-aminobenzenesulfonamide is reacted in the presence of ZnCl₂, as the Lewis acid catalyst, and wherein the N-[4-(aminosulfonyl)phenyl}-N′-cyanocarbamidic acid phenyl ester is isolated prior to reacting with 2,6-difluorobenzoic acid hydrazide, to yield the compound of formula (Ia).

Novel crystalline Form (Ia-2) may be characterized by its X-ray powder diffraction pattern, as listed in Table XRD-2 below.

TABLE XRD-2
Crystalline Form (Ia-2)

| Pos. [°2Theta] | d-spacing [Å] | Rel. Int. [%] |
|---|---|---|
| 7.71 | 11.46 | 7.63 |
| 12.87 | 6.88 | 10.11 |
| 13.74 | 6.44 | 17.65 |
| 14.21 | 6.23 | 9.45 |
| 14.74 | 6.01 | 100.00 |
| 15.26 | 5.81 | 21.35 |
| 15.44 | 5.74 | 12.37 |
| 16.32 | 5.43 | 8.25 |
| 16.69 | 5.31 | 9.10 |
| 16.77 | 5.29 | 7.43 |
| 18.15 | 4.89 | 23.77 |
| 19.02 | 4.67 | 9.27 |
| 19.45 | 4.56 | 28.96 |
| 19.67 | 4.51 | 28.55 |
| 20.29 | 4.37 | 15.34 |
| 20.55 | 4.32 | 15.89 |
| 20.77 | 4.27 | 11.87 |
| 21.27 | 4.17 | 16.03 |
| 21.47 | 4.14 | 11.42 |
| 22.06 | 4.03 | 10.74 |
| 22.88 | 3.88 | 5.46 |
| 24.69 | 3.60 | 40.20 |
| 25.46 | 3.50 | 12.51 |
| 25.78 | 3.45 | 14.85 |
| 26.21 | 3.40 | 17.42 |
| 26.72 | 3.33 | 24.18 |
| 27.17 | 3.28 | 15.01 |
| 27.47 | 3.25 | 9.40 |
| 28.50 | 3.13 | 17.31 |
| 28.78 | 3.10 | 25.55 |
| 29.34 | 3.04 | 6.50 |
| 29.91 | 2.98 | 9.85 |
| 31.62 | 2.83 | 7.16 |

In another embodiment of the present invention is a novel crystalline form of the compound of formula (Ia) characterized by the major X-ray diffraction peaks having a relative intensity of greater than or equal to about 10%, as listed in Table XRD-2 above. In another embodiment of the present invention is a novel crystalline form of the compound of formula (Ia) characterized by the major X-ray diffraction peaks having a relative intensity of greater than or equal to about 20%, as listed in Table XRD-2 above.

In an embodiment of the present invention is a novel crystalline CH₃SO₃H (methane sulfonyl) salt of the compound of formula (Ia). In another embodiment of the present invention is a novel crystalline CH₃SO₃H salt of the compound of formula (Ia) wherein the molar ratio of the compound of formula (Ia) to CH₃SO₃H is 1:1.

The CH₃SO₃H salt of the compound of formula (Ia) may be prepared by reacting the compound of formula (Ia) with CH₃SO₃H, preferably, in an amount equal to about 1 equivalent, in an organic solvent which can dissolve the compound of formula (Ia) and the CH₃SO₃H, and which is unreactive to the CH₃SO₃H, such as THF, dioxane, an alcohol (such as methanol, ethanol, and the like), and the like, preferably at a temperature of less than or equal to about room temperature.

Novel crystalline CH₃SO₃H salt of the compound of formula (Ia) may be characterized by its X-ray diffraction pattern as listed in Table XRD-3, below.

TABLE XRD-3
CH₃SO₃H Salt

| Pos. [°2Theta] | d-spacing [Å] | Rel. Int. [%] |
|---|---|---|
| 4.05 | 21.85 | 7.08 |
| 12.12 | 7.30 | 8.38 |
| 13.32 | 6.65 | 7.58 |
| 15.89 | 5.58 | 62.06 |
| 17.43 | 5.09 | 27.06 |
| 18.76 | 4.73 | 25.76 |
| 19.88 | 4.47 | 46.91 |
| 20.26 | 4.38 | 40.61 |
| 20.92 | 4.25 | 51.81 |
| 21.44 | 4.14 | 87.25 |
| 22.18 | 4.01 | 72.66 |
| 22.76 | 3.91 | 59.56 |
| 26.51 | 3.36 | 32.29 |
| 27.08 | 3.29 | 100.00 |
| 28.59 | 3.12 | 12.36 |
| 30.34 | 2.95 | 5.40 |
| 31.46 | 2.84 | 6.90 |
| 33.06 | 2.71 | 8.23 |
| 33.36 | 2.69 | 11.20 |
| 34.38 | 2.61 | 8.64 |

In another embodiment of the present invention is a novel crystalline CH₃SO₃H salt of the compound of formula (Ia) characterized by the major X-ray diffraction peaks having a relative intensity of greater than or equal to about 10%, as listed in Table XRD-3 above. In another embodiment of the present invention is a novel crystalline CH₃SO₃H salt of the compound of formula (Ia) characterized by the major X-ray diffraction peaks having a relative intensity of greater than or equal to about 20%, as listed in Table XRD-3 above.

In an embodiment of the present invention is a novel crystalline HCl (hydrochloric) salt of the compound of formula (Ia). In another embodiment of the present invention is a novel crystalline HCl salt of the compound of formula (Ia) wherein, the molar ratio of the compound of formula (Ia) to HCl is 1:1.

The HCl salt of the compound of formula (Ia) may be prepared by reacting the compound of formula (Ia) with HCl, preferably, in an amount equal to about 1 equivalent, in an organic solvent which can dissolve the compound of formula (Ia) and the the HCl, and which is unreactive to the HCl, such as THF, dioxane, an alcohol (such as methanol, ethanol, and the like), and the like, preferably at a temperature of less than or equal to about room temperature.

Novel crystalline HCl salt of the compound of formula (Ia) may be characterized by its X-ray diffraction pattern as listed in Table XRD4, below.

TABLE XRD-4
HCl Salt

| Pos. [°2Theta] | d-spacing [Å] | Rel. Int. [%] |
|---|---|---|
| 13.67 | 6.48 | 45.30 |
| 14.27 | 6.21 | 43.44 |
| 15.85 | 5.59 | 33.11 |
| 17.01 | 5.21 | 45.04 |
| 17.18 | 5.16 | 52.13 |
| 17.54 | 5.06 | 40.78 |
| 18.21 | 4.87 | 31.62 |
| 19.36 | 4.58 | 63.78 |
| 20.36 | 4.36 | 43.04 |
| 21.20 | 4.19 | 32.54 |
| 22.45 | 3.96 | 40.97 |
| 22.98 | 3.87 | 65.31 |
| 23.75 | 3.75 | 100.00 |
| 25.36 | 3.51 | 21.59 |
| 26.09 | 3.42 | 13.23 |
| 26.82 | 3.32 | 40.99 |
| 27.23 | 3.28 | 77.86 |
| 27.70 | 3.22 | 74.23 |
| 28.73 | 3.12 | 12.94 |
| 29.66 | 3.01 | 5.95 |
| 32.18 | 2.78 | 9.34 |
| 32.81 | 2.73 | 9.03 |
| 34.04 | 2.63 | 16.93 |

In yet another embodiment of the present invention is a novel crystalline HCl salt of the compound of formula (Ia) characterized by the major X-ray diffraction peaks having a relative intensity of greater than or equal to about 10%, as listed in Table XRD-4 above. In yet another embodiment of the present invention is a novel crystalline HCl salt of the compound of formula (Ia) characterized by the major X-ray diffraction peaks having a relative intensity of greater than or equal to about 20%, as listed in Table XRD-4 above.

In an embodiment of the present invention is a novel crystalline HBr (hydrobromic) salt of the compound of formula (Ia). In another embodiment of the present invention is a novel crystalline HBr salt of the compound of formula (Ia) wherein, the molar ratio of the compound of formula (Ia) to HBr is 1:1.

The HBr salt of the compound of formula (Ia) may be prepared by reacting the compound of formula (Ia) with HBr, preferably, in an amount equal to about 1 equivalent, in an organic solvent which can dissolve the compound of formula (Ia) and the HBr, and which is unreactive to the HBr, such as THF, dioxane, an alcohol (such as methanol, ethanol, and the like), and the like, preferably at a temperature of less than or equal to about room temperature.

Novel crystalline HBr salt of the compound of formula (Ia) may be characterized by its X-ray diffraction pattern as listed in Table XRD-5, below.

TABLE XRD-5
HBr Salt

| Pos. [°2Theta] | d-spacing [Å] | Rel. Int. [%] |
|---|---|---|
| 4.46 | 19.82 | 47.43 |
| 13.40 | 6.61 | 17.79 |
| 14.25 | 6.22 | 9.59 |
| 15.75 | 5.63 | 33.50 |
| 16.99 | 5.22 | 33.36 |
| 17.40 | 5.10 | 77.64 |
| 17.99 | 4.93 | 30.47 |
| 19.31 | 4.60 | 45.07 |
| 20.31 | 4.37 | 45.66 |
| 20.63 | 4.30 | 44.81 |
| 21.13 | 4.20 | 47.54 |
| 22.19 | 4.01 | 32.71 |
| 22.47 | 3.96 | 39.15 |
| 22.68 | 3.92 | 27.02 |
| 23.81 | 3.74 | 83.64 |
| 23.99 | 3.71 | 79.30 |
| 25.10 | 3.55 | 48.15 |
| 26.01 | 3.43 | 13.57 |
| 27.35 | 3.26 | 100.00 |
| 28.03 | 3.18 | 21.98 |
| 28.67 | 3.11 | 9.20 |
| 29.13 | 3.07 | 8.67 |
| 29.79 | 3.00 | 8.08 |
| 31.25 | 2.86 | 5.73 |
| 31.60 | 2.83 | 25.34 |
| 33.57 | 2.67 | 31.35 |

In yet another embodiment of the present invention is a novel crystalline HBr salt of the compound of formula (Ia) characterized by the major X-ray diffraction peaks having a relative intensity of greater than or equal to about 10%, as listed in Table XRD-5 above. In yet another embodiment of the present invention is a novel crystalline HBr salt of the compound of formula (Ia) characterized by the major X-ray diffraction peaks having a relative intensity of greater than or equal to about 20%, as listed in Table XRD-5 above.

In an embodiment of the present invention is a novel crystalline H₂SO₄ (sulfuric) salt of the compound of formula (Ia). In another embodiment of the present invention is a novel crystalline H₂SO₄ salt of the compound of formula (Ia) wherein, the molar ratio of the compound of formula (Ia) to H₂SO₄ is 1:0.5.

The H₂SO₄ salt of the compound of formula (Ia) may be prepared by reacting the compound of formula (Ia) with H₂SO₄, preferably, in an amount equal to about 1 equivalent, in an organic solvent which can dissolve the compound of formula (Ia) and the the H₂SO₄, and which is unreactive to the H₂SO₄, such as THF, dioxane, an alcohol (such as methanol, ethanol, and the like), and the like, preferably at a temperature of less than or equal to about room temperature.

Novel crystalline H₂SO₄ salt of the compound of formula (Ia) may be characterized by its X-ray diffraction pattern as listed in Table XRD-6, below.

TABLE XRD-6
H₂SO₄ Salt

| Pos. [°2Theta] | d-spacing [Å] | Rel. Int. [%] |
|---|---|---|
| 4.68 | 18.90 | 72.76 |
| 7.63 | 11.58 | 42.65 |
| 9.37 | 9.44 | 15.29 |
| 11.15 | 7.93 | 5.57 |
| 11.45 | 7.73 | 6.43 |
| 13.06 | 6.78 | 55.75 |
| 13.51 | 6.55 | 87.87 |
| 14.38 | 6.16 | 24.75 |
| 14.98 | 5.91 | 74.53 |
| 15.29 | 5.80 | 100.00 |
| 15.84 | 5.59 | 18.68 |
| 16.44 | 5.39 | 21.95 |
| 16.80 | 5.28 | 37.42 |
| 17.34 | 5.12 | 17.66 |
| 17.62 | 5.03 | 25.79 |
| 18.40 | 4.82 | 62.45 |
| 18.81 | 4.72 | 68.51 |
| 19.53 | 4.54 | 67.69 |
| 19.60 | 4.53 | 60.93 |
| 20.04 | 4.43 | 91.72 |
| 20.29 | 4.38 | 94.30 |
| 21.28 | 4.18 | 49.73 |
| 22.62 | 3.93 | 54.35 |
| 23.03 | 3.86 | 80.37 |
| 23.78 | 3.74 | 28.94 |
| 24.49 | 3.63 | 84.20 |
| 25.22 | 3.53 | 41.07 |
| 25.63 | 3.48 | 67.44 |
| 26.62 | 3.35 | 61.21 |
| 27.88 | 3.20 | 23.65 |
| 28.40 | 3.14 | 36.08 |
| 29.38 | 3.04 | 14.51 |
| 30.91 | 2.89 | 24.95 |
| 32.11 | 2.78 | 28.93 |
| 33.02 | 2.71 | 14.66 |
| 33.42 | 2.68 | 18.68 |
| 34.23 | 2.62 | 7.62 |

In yet another embodiment of the present invention is a novel crystalline H₂SO₄ salt of the compound of formula (Ia) characterized by the major X-ray diffraction peaks having a relative intensity of greater than or equal to about 10%, as listed in Table XRD-6 above. In yet another embodiment of the present invention is a novel crystalline H₂SO₄ salt of the compound of formula (Ia) characterized by the major X-ray diffraction peaks having a relative intensity of greater than or equal to about 20%, as listed in Table XRD-6 above.

The following Examples are set forth to aid in the understanding of the invention, and are not intended and should not be construed to limit in any way the invention set forth in the claims which follow thereafter.

### EXAMPLE 1

### N³-[(4-aminosulfonyl)phenyl]-1-(2′-thienoyl)-1H-1,2,4-triazole-3,5-diamine

### Compound #18

To a clean, dry reaction tube was sequentially charged 2-thiophenecarboxylic acid hydrazide (0.5951 g, 4.06 mmol), N-[4-(aminosulfonyl)phenyl]-N′-cyanocarbamidic acid phenyl ester. (1.2917 g, 4.00 mmol) and pyridine (10 mL). The reaction mixture was heated to 85° C. and allowed to stir for 22 h. After 3 hours a yellow solid precipitated. After 22 h the reaction mixture was cooled to 0° C. The solid that precipitated was isolated by filtration, washed with H₂O (15 mL), and dried in a vacuum oven at 60° C. for ca 48 h to yield N³-[(4-aminosulfonyl)phenyl]-1-(2′-thienoyl)-1H-1,2,4-triazole-3,5-diamine as a cream solid.

m.p.=283.0-287.0° C. (dec)

MS: [M+H]⁺=365, [M+Na]⁺=387, [2M+Na]⁺=751

¹H NMR (400 MHz, DMSO-d₆): δ 7.15 (2H, s), 7.34 (1H, dd), 7.80 (4H, s), 7.91 (2H, br s), 8.21 (1H, dd), 8.31 (1H, dd), 9.93 (1H, s) Elemental analysis for C₁₃H₁₂N₆O₂S₂; MW=364.41:

Calculated: C, 42.85; H, 3.32; N, 23.06; S, 17.60

Found: C, 43.32; H, 3.12; N, 22.68; S, 17.23.

### EXAMPLE 2

### N³-[(4-aminosulfonyl)phenyl]-1-(2′-furoyl)-1H-1,2,4-triazole-3,5-diamine

### Compound #17

To a clean, dry reaction tube was sequentially charged 2-furoic acid hydrazide (0.5214 g, 4.05 mmol), N-[4-(aminosulfonyl)phenyl]-N′-cyano-carbamidic acid phenyl ester (1.2919 g, 4.00 mmol) and pyridine (10 mL) to give a slightly turbid, pale yellow solution. The reaction mixture was heated to 85° C. and allowed to stir for 21.25 h. The reaction mixture was then cooled to room temperature and was added, dropwise to ca 300 mL of a vigorously stirred mixture of ice-H₂O. A pale yellow solid precipitated. The suspension was stirred for 20 min. The solid product was filtered and washed sequentially with IPA (ca 50 mL) and MTBE (ca 50 mL). The product was dried in a vacuum oven for 10 h at 90° C. to yield N³-[(4-aminosulfonyl)phenyl]-1-(2′-furanoyl)-1H-1,2,4-triazole-3,5-diamine as a cream solid.

m.p. >300° C.

MS: [M+H]⁺=349, [M+Na]⁺=371, [2M+Na]⁺=719

¹H NMR (400 MHz, DMSO-d₆): δ 6.88 (1H, dd), 7.15 (2H, s), 7.68 (2H, d), 7.76 (2H, d), 7.87 (2H, br s), 8.03 (1H, d), 8.18 (1H, s), 9.86 (1H, s) Elemental analysis for C₁₃H₁₂N₆O₄S; -MW=348.34-:

Calculated: C, 44.82; H, 3.47; N, 24.13; S, 9.21

Found: C, 44.62; H, 3.34; N, 23.89; S, 9.13.

### EXAMPLE 3

### N³-[(4-aminosulfonyl)phenyl]-1-(2′-methoxybenzoyl)-1H-1,2,4-triazole-3 5-diamine (Compound #3)

To a clean, dry reaction tube was sequentially charged 2-methoxybenzoic acid hydrazide (0.6864 g, 4.05 mmol), N-[4-(aminosulfonyl)phenyl]-N′-cyanocarbamidic acid phenyl ester (1.2913 g, 4.00 mmol) and pyridine (10 mL) to give a slightly turbid, cream solution. The reaction mixture was heated to 85° C. and allowed to stir. After 6.5 h, the reaction was complete as judged by HPLC analysis, the mixture was cooled to room temperature and added dropwise to ca 300 mL of a vigorously stirred mixture of ice-H₂O. A white solid precipitated. The suspension was stirred for 30 min. The solid product was filtered, washed with H₂O (2×30 mL) and dried in a vacuum oven at 80° C. for 10 h. The crude product was suspended in CH₃CN (ca 15-20 mL) at room temperature, filtered and dried to yield N³-[(4-aminosulfonyl)phenyl]-1-(2′-methoxybenzoyl)-1H-1,2,4-triazole-3,5-diamine as a white solid.

m.p. 217.0-221.5° C.

MS: [M+H]⁺=389, [M+Na]⁺=411, [2M+Na]⁺=799

¹H NMR (400 MHz, DMSO-d₆): δ 3.78 (3H, s), 7.07 (2H, s), 7.07 (1H, m), 7.20 (1H, dd), 7.48 (3H, m), 7.55 (3H, m), 7.80 (2H, br s), 9.70 (1H, s) Elemental analysis for C₁₆H₁₆N₆O₄S×0.09 H₂O; -MW=390.03-:

Calculated: C, 49.28; H, 4.18; N, 21.55; S, 8.22; H₂O, 0.42

Found: C, 49.00; H, 3.72; N, 21.59; S, 8.33; H₂O, 0.40.

### EXAMPLE 4

### N³-[(4-aminosulfonyl )phenyl]-1-(4′-hydroxybenzoyl)-1H-1,2,4-triazole-3,5-diamine (Compound #10)

To a clean, dry reaction tube was sequentially charged 4-hydroxybenzoic acid hydrazide (0.6285 g, 4.05 mmol), N-[4-(aminosulfonyl)phenyl]-N′-cyanocarbamidic acid phenyl ester (1.2915 g, 4.00 mmol) and pyridine (10 mL) to yield a white suspension. The reaction mixture was heated to 85° C. by which point solution was effected. After 20 h the reaction was cooled to room temperature and then added dropwise to ca 300 mL of a vigorously stirred mixture of ice-H₂O. A white solid precipitated. The suspension was stirred for 20-30 min. The solid product was filtered and washed sequentially with H₂O (ca 100 mL), IPA (ca 50 mL) and MTBE (ca 50 mL). Any precipitated solids in the filtrates were recovered and combined with the product and dried in a vacuum oven at 65° C. for 10 h to yield N³-[(4-aminosulfonyl )phenyl]-1-(4′-hydroxybenzoyl)-1H-1,2,4-triazole-3,5-diamine as a snow white solid.

m.p. >300° C.

MS: [M+H]⁺=375, [M+Na]⁺=397

¹H NMR (400 MHz, DMSO-d₆): δ 6.92 (2H, d), 7.12 (2H, s), 7.67 (4H, m), 7.79 (2H, br s), 8.17 (2H, d), 9.76 (1H, s), 10.45 (1H, br s) Elemental analysis for C₁₅H₁₄N₆O₄S; MW=374.38:

Calculated: C, 48.12; H, 3.77; N, 22.45; S, 8.57

Found: C, 48.06; H, 3.52; N, 22.09; S, 8.44.

### EXAMPLE 5

### N³-[(4-aminosulfonyl)phenyl]-1-(2′-chlorobenzoyl)-1H-1,2,4-triazole-3,5-diamine

### Compound #2

To a clean, dry reaction tube was sequentially charged 2-chlorobenzoic acid hydrazide (0.7053 g, 4.05 mmol), N-[4-(aminosulfonyl)phenyl]-N′-cyano-carbamidic acid phenyl ester (1.2910 g, 4.00 mmol) and pyridine (10 mL) to give a slightly turbid, pale yellow solution. The reaction mixture was heated to 85° C. by which point solution was effected. After 20 h the reaction mixture was cooled to room temperature and then added dropwise to ca 300 mL of a vigorously stirred mixture of ice-H₂O. A white solid precipitated. The suspension was stirred for 20-30 min. The solid product was filtered and washed sequentially with H₂O (Ca 100 mL), IPA (Ca 50 mL) and MTBE (Ca 50 mL). Any precipitated solids in the filtrates were recovered and combined with the product and dried in a vacuum oven for 10 h at 65° C. to yield N³-[(4-aminosulfonyl)phenyl]-1-(2′-chlorobenzoyl)-1H-1,2,4-triazole-3,5-diamine as a snow white solid.

m.p.=237.0-242.5° C.

MS: [M+H]⁺=393, [M+Na]⁺=415

¹H NMR (400 MHz, DMSO-d₆): δ 7.07 (2H, s), 7.46 (2H, d), 7.51-7.64 (3H, m), 7.55 (2H, d), 7.70 (1H, dd), 7.93 (2H, br s), 9.77 (1H, s) Elemental analysis for C₁₅H₁₃ClN₆O₃S; MW=392.83:

Calculated: C, 45.86; H, 3.34; N, 21.39; S, 8.16; Cl, 9.03

Found: C, 45.63; H, 3.07; N, 21.19; S, 8.18; Cl, 8.87.

### EXAMPLE 6

### N³-[(4-aminosulfonyl )phenyl]-1-(2′-bromobenzoyl)-1H-1,2,4-triazole-3,5-diamine

### Compound #4

To a clean, dry reaction tube was sequentially charged 2-bromobenzoic hydrazide (1.48 g, 6.88 mmol), N-[4-(aminosulfonyl)phenyl]-N′-cyanocarbamidic acid phenyl ester (2.0 g, 6.33 mmol) and pyridine (20 mL) to give a white suspension. The reaction mixture was heated to 85° C. by which point solution was effected. After 6 h the reaction mixture was cooled to room temperature and then added dropwise to ca 275 mL of a vigorously stirred mixture of 250 mL NH₄Cl solution and 25 mL of MeOH. A white solid precipitated. The suspension was stirred for 20-30 min. The solid product was filtered and washed with H₂O (ca 20 mL). The product was dried in a vacuum oven at 60-65° C. for 12 h. The crude product was suspended in methanol (60 mL) and stirred at room temperature overnight. The product was filtered, washed with methanol (10 mL) and dried in a vacuum oven at 60° C. to yield N³-[(4-aminosulfonyl)phenyl]-1-(2′-bromobenzoyl)-1H-1,2,4-triazole-3,5-diamine as a white solid.

HPLC purity: 99.4 A %

m.p.=246-248° C.

MS: [M+H]⁺=438.9, [M+Na]⁺=460.9

¹H NMR (300 MHz, DMSO-d₆): δ 7.09 (2H, s), 7.46 (2H, d), 7.47 (2H, m), 7.49 (2H, d), 7.67 (1H, dd), 7.76 (1H, dd), 7.94 (2H, br s), 9.79 (1H, s) Elemental analysis for C₁₅H₁₃BrN₆O₃S; MW=437.3:

Calculated: C, 41.20; H, 3.00; N, 19.22; S, 7.33; Br, 18.27

Found: C, 41.44; H, 2.94; N, 19.06; S, 7.24; Br, 18.44.

### EXAMPLE 7

### N³-[(4-aminosulfonyl )phenyl]-1-(3′-bromobenzoyl)-1H-1,2,4-triazole-3,5-diamine

### Compound #7

To a clean, dry reaction tube was sequentially charged 3-bromobenzoic hydrazide (1.48 g, 6.88 mmol), N-[4-(aminosulfonyl)phenyl]-N′-cyanocarbamidic acid phenyl ester (2.0 g, 6.33 mmol) and pyridine (20 mL) to give a white suspension. The reaction mixture was heated to 85° C. by which point solution was effected. After 6 h the reaction mixture was cooled to room temperature and then added dropwise to ca 275 mL of a vigorously stirred mixture of 250 mL NH₄Cl solution and 25 mL of MeOH. A white solid precipitated. The suspension was stirred for 20-30 min and the solid product was filtered and washed with H₂O (ca 20 mL). The product was dried in a vacuum oven at 60-65° C. for 12 h. The crude product was purified by suspending it in methanol (100 mL) and refluxing, after which time the suspension was cooled to 20-25° C. and filtered. This process was then repeated, the product was washed with methanol (10 mL) and dried in a vacuum oven at 70° C. for 48 h to yield N³-[(4-aminosulfonyl)phenyl]-1-(3′-bromobenzoyl)-1H-1,2,4-triazole-3,5-diamine as a white solid.

HPLC purity: 99.6 A %

m.p.=242-244° C.

MS: [M+H]⁺=438.9

¹H NMR (300 MHz, DMSO-d₆): δ 7.15 (2H, s), 7.56 (1H, m), 7.66 (4H, m), 7.91-7.92 (3H, m), 8.04 (1H, d), 8.49 (1H, s), 9.86 (1H, s) Elemental analysis for C₁₅H₁₃BrN₆O₃S; MW=437.3:

Calculated: C, 41.20; H, 3.00; N, 19.20; S, 7.33; Br, 18.27

Found: C, 41.14; H, 2.92; N, 19.07; S, 7.24; Br, 18.42.

### EXAMPLE 8

### N³-[(4-aminosulfonyl)phenyl]-1-(benzoyl)-1H-1,2,4-triazole-3,5-diamine

### Compound #1

To a clean, dry reaction tube was sequentially charged benzoic hydrazide (0.94 g, 6.88 mmol), N-[4-(aminosulfonyl)phenyl]-N′-cyanocarbamidic acid phenyl ester (2.0 g, 6.33 mmol) and pyridine (20 mL) to give a white suspension. The reaction mixture was heated to 85° C. by which point solution was effected. After 6 h the reaction mixture was cooled to room temperature and then added dropwise to ca 275 mL of a vigorously stirred mixture of 250 mL NH₄Cl solution and 25 mL of MeOH. A white solid precipitated. The suspension was stirred for 20-30 min and the solid product was filtered and washed with H₂O (ca 20 mL). The solid product was dried in a vacuum oven at 60-65° C. for 12 h. The crude product was suspended in methanol (180 mL) and stirred overnight. The product was filtered, washed with methanol (10 mL) and dried in a vacuum oven at 70° C. overnight to yield N³-[(4-aminosulfonyl)phenyl]-1-(benzoyl)-1H-1,2,4-triazole-3,5-diamine as a white solid.

HPLC purity: 99.4 A %

m.p. 354-356° C.

MS: [M+H]⁺=359.0, [M+Na]⁺=381.0

¹H NMR (300 MHz, DMSO-d₆): δ 7.12 (2H, s), 7.56-7.68 (7H, m), 7.89 (2H, br s), 8.14 (2H, m), 9.80 (1H, s) Elemental analysis for C₁₅H₁₄N₆O₃S; MW=358.4

Calculated: C, 50.27; H, 3.94; N, 23.45; S, 8.95

Found: C, 50.24; H, 3.95; N, 23.58; S, 9.05.

### EXAMPLE 9

### N³-[(4-aminosulfonyl)phenyl]-1-(3′-nitrobenzoyl)-1H-1,2,4-triazole-3,5-diamine

### Compound #5

To a clean, dry reaction tube was sequentially charged 3-nitrobenzoic hydrazide (1.26 g, 6.88 mmol), N-[4-(aminosulfonyl)phenyl]-N′-cyanocarbamidic acid phenyl ester (2.0 g, 6.33 mmol) and pyridine (20 mL) to give a light yellow suspension. The reaction mixture was heated to 85° C. by which point solution was effected. After 6 h the reaction mixture was cooled to room temperature and then added dropwise to ca 275 mL of a vigorously stirred mixture of 250 mL NH₄Cl solution and 25 mL of MeOH. A light yellow solid precipitated. The suspension was stirred for 20-30 min and the solid product was filtered and washed with H₂O (ca 20 mL). The crude product was purified by suspending it in a mixture of 150ml CH₃CN (150 mL) and THF (15 mL) at 60-70° C. The suspension was cooled to 20-25° C. and filtered. This purification process was then repeated, and the solid was washed with CH₃CN (20 mL) and dried at 60° C. in a vacuum oven for 48 h. to yield N³-[(4-aminosulfonyl)phenyl]-1-(3′-nitrobenzoyl)-1H-1,2,4-triazole-3,5-diamine as a light yellow solid.

HPLC purity: 99.5 A %

m.p. 260-262° C.

MS: [M+H]⁺=404.0 ; [M+Na]⁺=426.0

¹H NMR (300 MHz, DMSO-d₆): δ 7.17 (2H, s), 7.68 (4H, br s), 7.88 (1H, t), 7.97 (2H, br s), 8.50 (2H, m), 9.28 (1H, s), 9.88 (1H, s) Elemental analysis for C₁₅H₁₃N₇O₅S; MW=403.4:

Calculated: C, 44.66; H, 3.25; N, 24.31; S, 7.95

Found: C, 44.39; H, 3.26; N, 24.25; S, 8.03.

### EXAMPLE 10

### N³-[(4-aminosulfonyl)phenyl]-1-(4′-nitrobenzoyl )-1H-1,2,4-triazole-3,5-diamine

### Compound #9

To a clean, dry reaction tube was sequentially charged 4-nitrobenzoic hydrazide (1.26 g, 6.88 mmol), N-[4-(aminosulfonyl)phenyl]-N′-cyanocarbamidic acid phenyl ester (2.0 g, 6.33 mmol) and pyridine (20 mL) to give a yellow suspension. The reaction mixture was heated to 85° C. by which point solution was effected. After 6 h the reaction was cooled to room temperature and then added dropwise to ca 275 mL of a vigorously stirred mixture of 250 mL NH₄Cl solution and 25 mL of MeOH. A yellow solid precipitated. The suspension was stirred for 20-30 min and the solid product was filtered and washed with H₂O (ca 20 mL). The product was dried in a vacuum oven at 60-65° C. for 12 h. The crude product was suspended in refluxing THF (50 mL). The suspension was cooled to 20-25° C. and filtered. The solid was dried in a vacuum oven at 60° C. overnight to yield N³-[(4-aminosulfonyl)phenyl]-1-(4′-nitrobenzoyl)-1H-1,2,4-triazole-3,5-diamine as a light yellow solid.

HPLC purity: 96.8 A %.

m.p. 336-338° C.

MS: [M+H]⁺=404.0; [M+Na]⁺=426.0

¹H NMR (300 MHz, DMSO-d₆): δ 7.12 (2H, s), 7.58 (2H, d), 7.69 (2H, d), 7.97 (2H, br s), 8.31 (2H, d), 8.42 (2H, d), 9.85 (1H, s) Elemental analysis for C₁₅H₁₃N₇O₅S; MW=403.4:

Calculated: C, 44.66; H, 3.25; N, 24.31; S, 7.95

Found: C, 44.56; H, 3.30; N, 24.34; S, 7.57.

### EXAMPLE 11

### N³-[(4-aminosulfonyl)phenyl]-1-acetyl-1H-1,2,4-triazole-3,5-diamine

### Compound #16

To a clean, dry reaction tube was sequentially charged acetic hydrazide (0.52 g, 6.88 mmol), N-[4-(aminosulfonyl)phenyl]-N′-cyanocarbamidic acid phenyl ester (2.0 g, 6.33 mmol) and pyridine (20 mL) to give a white suspension. The reaction mixture was heated to 85° C. by which point solution was effected. After 6 h the reaction mixture was cooled to room temperature and then added dropwise to ca 275 mL of a vigorously stirred mixture of 250 mL NH₄Cl solution and 25 mL MeOH. A white solid precipitated. The suspension was stirred for 20-30 min and the solid product was filtered and washed with H₂O (ca 20 mL). The product was dried in a vacuum oven at 60-65° C. for 12 h. The crude product was suspended in EtOH (60 mL) and stirred at 20-25° C. overnight. The product was filtered, washed with EtOH (10 mL) and dried in a vacuum oven at 60° C. for 12 h to yield N³-[(4-aminosulfonyl)phenyl]-1-(acetyl)-1H-1,2,4-triazole-3,5-diamine as a white solid.

m.p. 334-336° C.

MS: [M+H]⁺=297.0

¹H NMR (300 MHz, DMSO-d₆): δ 2.52 (3H, s), 7.13 (2H, s), 7.62 (2H, br s), 7.69 (4H, s), 9.72 (s, 1H) Elemental Analysis for C₁₀H₁₂N₆O₃S; MW=296.3:

Calculated: C, 40.53; H, 4.08; N, 28.36; S, 10.82

Found: C, 40.35; H, 3.86; N, 28.25; S, 11.04.

### EXAMPLE 12

### N³-[(4-aminosulfonyl)phenyl]-1-(4′-methoxybenzoyl)-1H-1,2,4-triazole-3,5-diamine (Compound #12)

To a clean, dry reaction tube was sequentially charged 4-methoxybenzoic hydrazide (0.80 g, 4.73 mmol), N-[4-(aminosulfonyl)phenyl]-N′-cyanocarbamidic acid phenyl ester (1.30 g, 4.02 mmol) and pyridine (10 mL). After stirring at room temperature for 5-10 min solution was effected after which time the reaction mixture was heated to 85° C. and stirred at 85° C. for 3 h. The reaction mixture was cooled to room temperature and then added dropwise to ca 150 mL of a vigorously stirred mixture of ice-saturated NaCl solution. A white solid precipitated. The suspension was stirred for 20-30 min and the solid product was filtered, washed with H₂O (ca 100 mL) and dried in a vacuum oven at 60-65° C. for 12 h. The crude product was suspended in MeOH (50 mL) and stirred at 20-25° C. overnight. The product was filtered, washed with MeOH (10 mL) and dried in a vacuum oven at 70° C. for 12 h to yield N³-[(4-aminosulfonyl)phenyl]-1-(4′-methoxybenzoyl)-1H-1,2,4-triazole-3,5-diamine as a white solid.

m.p. 244.5-247.5° C.

MS: [M+H]⁺=389.0, [M+H]⁺=411

¹H NMR (300 MHz, DMSO-d₆): δ 3.89 (3H, s), 7.13 (2H, s), 7.14 (2H, d), 7.64 (2H, d), 7.71 (2H, d), 7.85 (2H, br s), 7.69 (4H, s), 8.25 (2H, d), 9.80 (s, 1H) Elemental Analysis for C₁₆H₁₆N₆O₄S×0.1 H₂O; MW=390.2:

Calculated: C, 49.25; H, 4.18; N, 21.54; S, 8.22; H₂O, 0.46

Found: C, 48.92; H, 3.93; N, 21.34; S, 8.00; H₂O, 0.51.

### EXAMPLE 13

### N³-[(4-aminosulfonyl)phenyl]-1-(4′-phenylbenzoyl)-1H-1,2,4-triazole-3,5-diamine (Compound #11)

To a clean, dry reaction tube was sequentially charged 4-phenylbenzoic hydrazide (0.99 g, 4.65 mmol), N-[4-(aminosulfonyl)phenyl]-N′-cyanocarbamidic acid phenyl ester (1.30 g, 4.02 mmol) and pyridine (10 mL). After stirring at room temperature for 5-10 min solution was effected after which time the reaction mixture was heated to 85° C. and stirred at 85° C. for 3 h. The reaction mixture was cooled to room temperature whereupon a solid precipitated. The cream suspension was reheated to ca 60° C. to effect solution, which was then added dropwise to ca 150 mL of a vigorously stirred mixture of ice-saturated NaCl solution. A pale yellow solid precipitated. The suspension was stirred for 20-30 min and the solid product was filtered and washed with H₂O (ca 100 mL) and then air dried. The crude product was dissolved in DMSO (5 mL) and purified on a silica gel column (30 g) using a mixture of ethyl acetat/n-heptane (80/20). Product containing fractions were combined and evaporated. The resulting solid was suspended in water (9 mL) and stirred at 55° C. for 2 h. The suspension was then cooled to room temperature and filtered. The solid was washed with water (15 mL) and dried in a vacuum oven at 90° C. for 36 h to yield N³-[(4-aminosulfonyl)phenyl]-1-(4′-phenylbenzoyl)-1H-1,2,4-triazole-3,5-diamine as a white solid.

m.p. >260° C.

MS: [M+H]⁺=435.0

¹H NMR (300 MHz, DMSO-d₆): δ 7.11 (2H, s), 7.46 (1H, t), 7.54 (2H, t), 7.66 (2H, d), 7.71 (2H, d), 7.81 (2H, d), 7.91 (2H, d), 7.93 (2H, br s), 8.29 (2H, d),9.83(s,1H) Elemental Analysis for C₂₁H₁₈N₆O₃S×0.55 H₂O; MW=444.39

Calculated: C, 56.76; H, 4.33: N, 18.91; S, 7.22; H₂O, 2.23

Found: C, 56.50; H, 4.16: N, 18.51; S, 7.23; H₂O, 2.31.

### EXAMPLE 14

### N³-[(4-aminosulfonyl)phenyl]-1-(4′-chlorobenzoyl)-1H-1,2,4-triazole-3,5-diamine

### Compound #28

To a clean, dry reaction tube was sequentially charged 4-chlorobenzoic hydrazide (0.83 g, 4.79 mmol), N-[4-(aminosulfonyl)phenyl]-N′-cyanocarbamidic acid phenyl ester (1.30 g, 4.02 mmol) and pyridine (10 mL). After stirring at room temperature for 5-10 min solution was effected after which time the reaction mixture was heated to 85° C. and stirred at 85° C. for 3 h. The reaction mixture was cooled to room temperature whereupon a solid precipitated. The yellow suspension was reheated to ca 60° C. to effect solution, which was then added dropwise to ca 150 mL of a vigorously stirred mixture of ice-saturated NaCl solution. A white solid precipitated. The suspension was stirred for 20-30 min and the solid product was filtered and washed with H₂O (ca 100 mL) and air dried. The crude product was dissolved in DMSO (5 mL) and purified on a silica gel column (35 g) using a mixture of ethyl acetate/n-heptane (80/20). Product containing fractions were combined and evaporated. The resulting oily solid containing residual DMSO was suspended in water (10 mL) and stirred at 40° C. for 14 h. The suspension was cooled to room temperature and filtered. The solid was then washed with water (20 mL). The product was dried in a vacuum oven at 130° C. for 60 h to yield N³-[(4-aminosulfonyl)phenyl]-1-(4′-chlorobenzoyl)-1H-1,2,4-triazole-3,5-diamine as a white solid.

m.p. >260° C.

MS: [M+H]+=393.0

¹H NMR (300 MHz, DMSO-d₆): δ 7.14 (2H, s), 7.62 (2H, d), 7.69 (4H, m), 7.92 (2H, br s), 8.19 (2H, d), 9.83 (s; 1H) Elemental Analysis for C₁₅H₁₃ClN₆O₃S×0.25 H₂O; MW=397.33

Calculated: C, 45.34; H, 3.42: N, 21.15; S, 8.07; Cl, 8.92; H₂O, 1.13

Found: C, 45.44; H, 3.19; N, 20.45; S, 7.98; Cl, 9.39; H₂O, 1.42.

### EXAMPLE 15

### N³-phenyl-1-(4′-methylbenzoyl)-1,2,4-triazole-3,5-diamine

### Compound #25

A solution of aniline (0.3845 g, 4.10 mmol) and diphenylcyano-carbonimidate (0.9830 g, 4.00 mmol) in pyridine (15 mL) was stirred at room temperature for 1 h at which time HPLC analysis showed the reaction to be complete. 4-Methylbenzoic acid hydrazide (0.6074 g, 4.00 mmol) was added and the clear yellow solution was heated to 85° C. The reaction mixture was stirred at 85° C. for 9 h after which time the reaction mixture was cooled to room temperature and added dropwise to ca 200 mL of a vigorously stirred mixture of ice-H₂O. A white solid precipitated. The suspension was stirred for 1 h and then filtered. The solid was washed with H₂O (ca 100 mL) and then air dried for several hours. The crude product was suspended in MeOH (30 mL) and stirred for several hours at room temperature. The suspension was filtered and the solid was washed with MeOH and dried in a vacuum oven at 100° C. for 12 h to yield N³-phenyl-1-(4′-methylbenzoyl)-1,2,4-triazole-3,5-diamine as a white solid.

m.p. 222.5-224.0° C.

MS: [M+H]⁺=294, [M+Na]⁺=317

¹H NMR (300 MHz, DMSO-d₆): δ 2.49 (3H, s), 6.85 (1H, br t), 7.23 (2H, br t), 7.38 (2H, d), 7.54 (2H, d), 7.82 (2H, br s), 8.13 (2H, d), 9.31 (s,1H) Elemental Analysis for C₁₆H₁₅N₅O; MW=293.33:

Calculated: C, 65.52; H, 5.15; N, 23.88

Found: C, 65.26; H, 5.03; N, 23.90.

### EXAMPLE 16

### N³-phenyl-1-(2′methoxybenzoyl)-1,2,4-triazole-3,5-diamine

### Compound #29

A solution of aniline (0.3845 g, 4.10 mmol) and diphenylcyano-carbonimidate (0.9817 g, 4.00 mmol) in pyridine (15 mL) was stirred at room temperature for 1 h at which time HPLC analysis showed the reaction to be complete. 2-Methoxybenzoic acid hydrazide ( 0.6855 g, 4.00 mmol) was added and the resulting yellow solution was heated to 85° C. and stirred at 85° C. for 4 h. After 4 h the reaction mixture was cooled to room temperature and added dropwise to ca 200 mL of a vigorously stirred mixture of ice-H₂O. A white solid precipitated. The suspension was stirred for 0.5 h and then filtered. The solid was washed with H₂O (ca 100 mL)and then air dried for 1 h. The crude product was suspended in CH₃CN (5 mL) and stirred at room temperature overnight. The suspension was filtered, the solid was washed with CH₃CN, and then dried in a vacuum oven at 70° C. for 5 h to yield N³-phenyl-1-(2′-methoxybenzoyl)-1,2,4-triazole-3,5-diamine as a white solid.

m.p. 89.5-94.0° C.

MS: [M+H]⁺=310, [M+Na]⁺=332

¹H NMR (300 MHz, DMSO-d₆): δ 3.77 (3H, s), 6.77 (1H, t), 7.03-7.19 (2H, m), 7.09 (2H, d), 7.37 (2H, d), 7.45-7.55 (2H, m), 7.75 (2H, br s), 9.19 (s, 1H)

| | | |
|---|---|---|
| HRMS: For C₁₆H₁₅N₅O₂: | Calculated: | 310.1299 |
| | Found: | 310.1306 |

### EXAMPLE 17

### N³-phenyl-1-(3′-methoxybenzoyl)-1,2,4-triazole-3,5-diamine

### Compound #30

A solution of aniline (0.3845 g, 4.10 mmol) and diphenylcyano-carbonimidate (0.9822 g, 4.00 mmol) in pyridine (15 mL) was stirred at room temperature for 1 h at which time HPLC analysis showed the reaction to be complete. 3-Methoxybenzoic acid hydrazide (0.6794 g, 4.00 mmol) was added to yield a light tan solution which was heated to 85° C. and stirred at 85° C. for 4 h. After 4 h the reaction was cooled to room temperature and then added dropwise to ca 200 mL of a vigorously stirred mixture of ice-H₂O. A yellow solid precipitated. The suspension was stirred for 0.5 h and then filtered. The solid was washed with H₂O (ca 100 mL) and then air dried for 1 h. The crude product was washed with CH₃CN (2×25 mL), MTBE (25 mL) and the product was dried in a vacuum oven at 40° C. for 12 h to yield N³-phenyl-1-(3′-methoxybenzoyl)-1,2,4-triazole-3,5-diamine as a pale yellow solid.

m.p. 174-184° C.

MS: [M+H]⁺=310, [M+Na]⁺=332

¹H NMR (300 MHz, DMSO-d₆): δ 3.85 (3H, s), 6.84 (1H, t), 7.18-7.25 (3H, m), 7.46-7.55 (3H, m), 7.72 (1H, s), 7.84 (3H, br s), 9.34 (s, 1H)

| | | |
|---|---|---|
| HRMS: For C₁₆H₁₅N₅O₂: | Calculated: | 310.1299 |
| | Found: | 310.1302 |

### EXAMPLE 18

### N³-phenyl-1-(2-furoyl)-1,2,4-triazole-3,5-diamine

### Compound #31

A solution of aniline (0.3845 g, 4.10 mmol) and diphenylcyano-carbonimidate (0.9825 g, 4.00 mmol) in pyridine (15 mL) was stirred at room temperature for 2 h at which time HPLC analysis showed the reaction to be complete. 2-Furoic acid hydrazide (0.5144 g, 4.00 mmol) was added to yield an amber solution which was heated to 85° C. and stirred at 85° C. for 23.5 h. After 23.5 h the reaction mixture was cooled to room temperature and then added dropwise to ca 200 mL of a vigorously stirred mixture of ice-H₂O. A tan solid precipitated. The suspension was stirred for 1 h and then filtered. The solid was washed with H₂O (ca 100 mL)and was air dried for 1 h. The crude product was recrystallized from CH₃CN/H₂O (1:1), filtered, and dried in a vacuum oven at 45° C. for 12 h to yield N³-phenyl-1-(2-furoyl)-1,2,4-triazole-3,5-diamine as a cream solid.

m.p. 201.0-202.0° C.

MS: [M+H]⁺=270, [M+Na]⁺=292

¹H NMR (300 MHz, DMSO-d₆): δ 6.86-6.92 (2H,m), 7.31 (2H, t), 7.57 (2H, d), 7.82 (2H, br s), 8.05 (1H, d), 8.17 (1H, d), 9.39 (s, 1H) Elemental Analysis for C₁₃H₁₁N₅O₂; MW=269.26

Calculated: C, 57.99; H, 4.12; N, 26.01

Found: C, 58.01; H, 3.94; N, 25.91.

### EXAMPLE 19

### N-[4-(aminosulfonyl)phenyl]-N′-cyanocarbamidic acid phenyl ester

A solution of diphenylcyanocarbonimidate (DPCCl) (10.0 g, 42.0 mmol) in THF (150 mL) at about 20-25° C. was treated with 0.5M ZnCl₂ in THF (6.1 mL, 3.0 mmol). The flask containing the reaction mixture was sealed and the reaction mixture was stirred overnight at about 20-25° C. After stirring overnight, 4-aminobenzenesulfonamide (7.2 g, 41.8 mmol) was added to the reaction mixture. The reaction mixture was then heated to reflux and held at this temperature, with stirring, for 10 h. A solid precipitated from the reaction mixture during this time. After 10 h, the reaction mixture was cooled to about 0-5° C., the solid was collected by filtration, washed with THF (20 mL) and dried in a vacuum oven at about 60-70° C. overnight to yield N-[4-(aminosulfonyl)phenyl]-N′-cyanocarbamidic acid phenyl ester as a white solid.

mp >250° C.

[M+H]⁺=317.0, [M+Na]⁺=339.0

¹H NMR (400 MHz, DMSO): δ 7.34 (5H, m), 7.46-7.(2H, m), 7.66(2H, d), 7.85 (2H, d)7. ),11.13 (1H, s) Elemental Analysis for C₁₄H₁₂N₄O₃S; MW=316.34:

Calculated: C, 53.16; H, 3.82; N, 17.71; S, 10.14

Found: C, 52.39; H, 3.67; N, 17.32; S, 9.87.

KF=0.30% H₂O

### EXAMPLE 20

### Preparation of N-[4-(aminosulfonyl)phenyl]-N′-cyanocarbamidic acid phenyl ester

A solution of diphenylcyanocarbonimidate (DPCCl) (875.0 g, 3.67 mol) in DME (12.0 L) at about 20-25° C. was treated with 0.5M ZnCl₂ in THF (510.0 mL, 0.255 mol). The flask containing the reaction mixture was sealed and the reaction mixture was stirred overnight at about 20-25° C. After stirring overnight, 4-aminobenzenesulfonamide (668.0 g, 3.88 mol) was added and the reaction mixture was then heated to reflux and held at reflux temperature, with stirring, for 10 h. A solid precipitated from the reaction mixture during this time. After 10 h the reaction mixture was cooled to about 0-5° C., the solid was collected by filtration, washed with DME (700 mL) and dried in a vacuum oven at about 50-70° C. overnight to yield N-[4-(aminosulfonyl)phenyl]-N′-cyano-carbamidic acid phenyl ester. This material was used in subsequent steps without further characterization.

HPLC purity: 93.7 A %, 93.4 wt %

KF 0.46% H₂O

### EXAMPLE 21

### N-[4-(aminosulfonyl)phenyl]-N′-cyanocarbamidic acid phenyl ester

A solution of 4-aminobenzenesulfonamide (850 g, 4.89 mol) in pyridine (4.0 L) was stirred and cooled in an ice bath as diphenylcyanocarbonimidate (DPCCl) (600 g, 2.45 mol) was added. The mixture was stirred at <30° C., while the solids dissolved. A second portion of diphenylcyanocarbonimidate (DPCCl) (600 g, 2.45 mol) was added followed by pyridine (0.77 L). The mixture was stirred at <30° C., while the solids dissolved. After 3.5 h stirring, the reaction was judged to be complete by HPLC analysis (<1% of DPCCl remaining) during which time the reaction mixture became a thick white suspension. Methyl tert-butyl ether (10.0 L) was then added to the reaction mixture and the suspension was stirred and cooled to about 0-5° C. The solid was isolated by filtration, washed with methyl tert-butyl ether (4.0 L), and dried in a vacuum oven overnight at about 80° C./29.5″ to yield N-[4-(aminosulfonyl)phenyl]-N′-cyanocarbamidic acid phenyl ester as a white solid.

HPLC purity: 96.4 wt %

[M+H]⁺=317.0, [M+Na]⁺=339.0 ¹H NMR (400 MHz, DMSO): δ 7.30-7.50 (5H, m,), 7.65 (2H, d), 7.85 (2H, d), 11.14 (1H, s) Elemental analysis for C₁₄H₁₂N₄O₃S; MW=316.34:

Calculated: C, 53.16; H, 3.82; N, 17.71; S, 10.14.

Found: C, 53.10; H, 3.65; N, 17.52; S, 9.86.

### EXAMPLE 22

### N³-[(4-aminosulfonyl)phenyl]-1-(2′,6′-difluorobenzoyl)-1H-1,2,4-triazole-3,5-diamine (Compound (Ia))

A mixture of N-[4-(aminosulfonyl)phenyl]-N′-cyanocarbamidic acid phenyl ester (2.0 g, 6.33 mmol), 2,6-difluorobenzoyl hydrazide (1.2 g, 6.98 mmol) and DMF (10 mL) was stirred at about 20-30° C. until a solution was achieved. The reaction mixture was then heated to 110° C. The reaction was judged to be complete by HPLC after 3.5 hours (<1% O-phenylisourea remaining). The reaction mixture was cooled to about 20-30° C. and then quenched into water (100 mL). The crude solid was filtered, dissolved in a small volume of DMF (1 mL) and chromatographed on silica gel using EtOAc as the eluent. Evaporation of the EtOAc fractions yielded N³-[(4-aminosulfonyl)-phenyl]-1-(2′,6′-difluorobenzoyl)-1H-1,2,4-triazole-3,5-diamine as a light yellow solid.

¹H NMR (300 MHz, DMSO): δ 7.20 (2H, s), 7.35 (2H, t), 7.45 (2H, d), 7.55 (2H, d), 7.75 (1H, m,), 8.05 (2H, br s ), 9.85 (1H, s)

### EXAMPLE 23

### N³-[(4-aminosulfonyl )phenyl]-1-(2′,6′-difluorobenzoyl)-1H-1,2,4-triazole-3,5-diamine (Compound (Ia))

STEP A:

A mixture of diphenylcyanocarbonimidate (DPCCl) (100.0 g, 0.42 mol), 4-aminobenzenesulfonamide (73.0 g, 0.42 mol) and pyridine (350 mL) was stirred at about 20-30° C. for 10 h. The resulting white suspension was then treated with 2,6-difluorobenzoylhydrazide (84.0 g, 0.49 mol) and then heated to about 70-80° C. All starting materials dissolved by about 40-50° C. to yield a light brown solution. After 4 h, the reaction was complete as judged by HPLC analysis (<2% of residual O-phenylisourea).

The light brown solution was then cooled to about 20-25° C. and quenched by addition to 7.5% aqueous NH₄Cl solution (1800 mL). The temperature of the quench mixture was maintained at about 55-60° C. A solid precipitated during the quench. Methanol (100 mL) was then added to the, reaction mixture which was stirred at 55-60° C. for 20 minutes and then the pale yellow suspension was cooled to about 20-25° C. The solid was filtered, washed with water (1000 mL) and dried for 60 h in a vacuum oven at about 90-100° C. to yield the crude product. This material was used without further characterization for Step B.

KF=0.86% H₂O

STEP B:

The crude solid was stirred in THF (350 mL) for 30 min at about 55-60° C. and filtered through a Celite pad to remove a small amount of insoluble material. The Celite pad was washed with 50-70 mL of THF and the combined clear, yellow filtrate and washes were concentrated to a volume of 150 mL at about 60-70° C. During the concentration the product began to crystallize. Acetonitrile (600 mL) was added to further crystallize the product. The resulting white suspension was cooled to about 0-5° C. and the re-crystallized product was filtered, washed with acetonitrile (100 mL) and dried overnight. The product was slurried in water (1800 mL) and 50 mL of MeOH. The white slurry was heated to 100° C. and water (450 mL) was distilled at atmospheric pressure to remove residual acetonitrile. The suspension was then cooled to 20° C. and filtered. The solid was washed with water (200 mL) and dried in a vacuum oven overnight at about 90° C. to yield N³-[(4-aminosulfonyl)phenyl]-1-(2′,6′-difluorobenzoyl)-1H-1,2,4-triazole-3,5-diamine as a white solid.

HPLC purity: 98.1 A %, 95.9 wt %

[M+H]⁺=395.0 Elemental analysis for C₁₅H₁₂F₂N₆O₃S: MW=394.36

Calculated: C, 45.68; H, 3.07; F, 9.64; N, 21.31; S, 8.13

Found: C, 45.67; H, 2.87; F, 9.79; N, 21.00; S, 7.76

KF=0.28% H₂O

PXRD, IR and DSC all showed this material to be crystalline polymorph Form (Ia-1).

### EXAMPLE 24

### N³-[(4-aminosulfonyl)phenyl]-1-(2′,6′-difluorobenzoyl)-1H-1,2,4-triazole-3,5-diamine (Compound (Ia))

The starting N-[4-(aminosulfonyl)phenyl]-N′-cyanocarbamidic acid phenyl ester was prepared and isolated from pyridine as described in Example 9, Step A above.

A mixture of N-[4-(aminosulfonyl)phenyl]-N′-cyanocarbamidic acid phenyl ester (1350.0 g, 4.09 mol) of, 2,6-difluorobenzoyl hydrazide (732.0 g, 4.25 mol) and pyridine (6.75 L) was stirred at about 20-30° C. until a solution was achieved. The reaction mixture was then heated to about 85-90° C. and held at this temperature for 6 h after which time the reaction was judged to be complete by HPLC analysis.

The light brown solution was then cooled to about 20-30° C. and quenched into 7.4% aqueous NH₄Cl solution (35.0 L) while maintaining the quench solution at about 50-60° C. A solid was observed to precipitate during the quench. Methanol (1.35 L) was then added to the reaction mixture and the resulting pale yellow suspension was cooled to about 20-25° C. The solid was filtered and washed with water (5.4 L) and dried overnight in a vacuum oven at about 85-95° C. to yield a crude solid.

KF=1.45% H₂O

The crude solid was stirred in THF (5.0 L) for 30 min at about 20-25° C. and filtered to remove a small amount of insoluble material. The clear, yellow filtrate was concentrated to a volume of 3.0 L at about 60-70° C., at which point acetonitrile (9.8 L) was added to crystallize the product. The white suspension was cooled to about 0-5° C. and filtered. The product was washed with acetonitrile (2.0 L) and then slurried in water (13.5 L). The white suspension was heated to 100° C. and water (2.7 L) was distilled off to remove residual acetonitrile. The suspension was then cooled to 20° C. and filtered to yield a white solid.

The white solid was dried overnight and then dissolved in THF (13.7 L). 37% Hydrochloric acid (304 mL, 4.29 mol) was then added to the solution of white solid in THF, whereupon the HCl salt of the title compound precipitated almost immediately. This salt was filtered, dried and then reslurried in water (13.7 L). The resulting white suspension was stirred at ambient temperature until the pH showed no further change (pH meter). At this point, the suspension was filtered and the resulting white solid was dried in a vacuum oven at 90° C. overnight to yield N³-[(4-aminosulfonyl)phenyl]-1-(2′,6′-difluorobenzoyl)-1H-1,2,4-triazole-3,5-diamine as a white solid.

HPLC purity: 99.99 wt %

m.p. 237-239° C.

MS: [M+H]⁺=395, [M+Na]⁺=417

¹H NMR (500 MHz, DMSO): δ 7.09(2H, s), 7.34 (2H, t), 7.47 (2H, d), 7.58 (2H, d), 7.71 (1H, m), 8.01 (2H, br s ), 9.84 (1H, s) Elemental analysis for C₁₅H₁₂F₂N₆O₃S×0.1 H₂O:

Calculated: C, 45.48; H, 3.10; F, 9.59; N,21.21; S, 8.09; H₂O, 0.45

Found: C, 45.33; H, 2.99; F, 9.59; N, 21.05; S, 7.76; H₂O, 0.38.

PXRD, IR and DSC all show this material to be crystalline polymorph Form (Ia-1).

### EXAMPLE 25

### Preparation of N³-[(4-aminosulfonyl)phenyl]-1-(2′,6′-difluorobenzoyl)-1H-1,2,4-triazole-3,5-diamine (Compound (Ia))

A series of experiments was run to determine the effect of solvent on the HPLC determined yield of the title product. The general procedure for the experiments was as follows. A mixture of N-[4-(aminosulfonyl)phenyl]-N′-cyanocarbamidic acid phenyl ester (0.5 g, 1.60 mmol) and 2,6-difluorobenzoyl hydrazide (0.3 g, 1.74 mmol) in 15ml of the selected solvent (see Table 3 below) was stirred and heated to about 80-85° C. The reaction mixture was maintained at about 80-85° C. overnight. After cooling the reaction mixture to about 20-25° C. an aliquot was removed for HPLC analysis. An HPLC sample was prepared by diluting the aliquot with acetonitrile and water (50/50) to determine % conversion to the title compound, with results as listed in Table 3 below.

TABLE 3
Effect of Solvent on Title Product Yield^{a}

| Solvent | MeOH | THF | DME | IPA | MeCN |
|---|---|---|---|---|---|
| % Yield | 2.3 | 3.0 | 1.0 | 0.3 | 1.8 |

^{a}HPLC A % conversion to title compound

### EXAMPLE 26

### Preparation of N³-[(4-aminosulfonyl)phenyl]-1-(2′,6′-difluorobenzoyl)-1H-1,2,4-triazole-3,5-diamine (Compound (Ia))

A series of experiments was run to determine the effect of solvent and base on the HPLC determined yield of the title product. The general procedure for the experiments was as follows. A mixture of N-[4-(aminosulfonyl)phenyl]-N′-cyanocarbamidic acid phenyl ester (0.5 g, 1.60 mmol) and 2,6-difluorobenzoyl hydrazide (0.3g, 1.74 mmol) in 15 ml of the selected solvent (See Table 4 below) was stirred during the addition of (2.08 mmol, 1.3 equivalents) of the selected base (See Table 4 below). The reaction mixture was heated to about 80-85° C. and maintained at this temperature for 6h. After cooling the reaction mixture to about 20-25° C. an aliqout was removed for HPLC analysis. An HPLC sample was prepared by diluting the aliquot with acetonitrile and water (50/50) to determine % conversion to the title compound, with results as listed in Table 4 below.

TABLE 4
Effect of Solvent and Base on Title Product Yield^{a,b}

| | | | | | |
|---|---|---|---|---|---|
| Solvent | MeOH | THF | DME | IPA | MeCN |

base = K₂CO₃

| | | | | | |
|---|---|---|---|---|---|
| % yield | 3.2 | 31.0 | 17.5 | 13.6 | 3.0 |

base = Cs₂CO₃

| | | | | | |
|---|---|---|---|---|---|
| % yield | 0.0 | 3.6 | 3.6 | 0.9 | 0.2 |

base = TEA

| | | | | | |
|---|---|---|---|---|---|
| % yield | 5.0 | 5.4 | 5.5 | 1.1 | 20.7^{c} |

base = DIPEA

| | | | | | |
|---|---|---|---|---|---|
| % yield | 1.8 | 2.8 | 1.6 | 2.7 | 16.1^{d} |

base = Pyridine

| | | | | | |
|---|---|---|---|---|---|
| % yield | 5.9 | 6.9 | 4.2 | 2.2 | 11.0 |

base = KOH^{e}

| | | |
|---|---|---|
| % yield | 14.5 | 9.3 |

base = NaOH pellets

| | | |
|---|---|---|
| % yield | 5.0 | 10.0 |

^{a}HPLC A % conversion to the title compound
^{b}Varying amounts of isourea exchange products and decomposition were observed in all cases except for those using pyridine
^{c}HPLC analysis showed ˜3% of another regioisomer
^{d}HPLC analysis showed ˜1.4% of another regioisomer
^{e}pellets

### EXAMPLE 27

### CH₃SO₃H Salt of 4-[5-Amino-1-(2,6-difluoro-benzoyl)-1H-[1,2,4]triazol-3-ylamino]-benzenesulfonamide

A mixture of 4-[5-Amino-1-(2,6-difluoro-benzoyl)-1H-[1,2,4]triazol-3-ylamino]-benzenesulfonamide (2.0 gm) in THF (20 ml) was stirred at room temperature to form a solution after which, CH₃SO₃H (0.49 g, 0.95 eq.) was added. The CH₃SO₃H salt precipitated rapidly. The resulting suspension was stirred for an additional 20 minutes at ambient temperature and the solid was collected by filtration. The filter cake was washed with THF (4 mL) and dried in a vacuum oven at 90° C. 3 days to yield the title compound as a white solid which contained 0.7% CH₃CN.

m.p.=279-281° C.

MS: [M+H]⁺=395 (free base)

¹HNMR (500 MHz, DMSO-d₆): δ 2.43, (3H, s), 7.08 (2H, br s), 7.34 (2H, t), 7.46 (2H, d), 7.58 (2H, d), 7.72 (1H, m), 8.01 (2H, br s), 9.84 ( s, 1H). Elemental Analysis for C₁₆H₁₆F₂N₆O₆S₂, MW=490.47:

Calculated: C, 39.18; H, 3.29; F, 7.75; N, 17.13; S, 13.08

Found: C, 39.26; H, 3.12; F, 7.72; N, 17.03; S, 12.98.

### EXAMPLE 28

### HCl Salt of 4-[5-Amino-1-(2,6-difluoro-benzoyl)-1H-[1,2,4]triazol-3-ylamino]-benzenesulfonamide

A mixture of 4-[5-Amino-1-(2,6-difluoro-benzoyl)-1H-[1,2,4]triazol-3-ylamino]-benzenesulfonamide (2.0 gm) in THF (20 ml) was stirred at room temperature to effect a solution after which 10N HCl (0.48 ml, 0.95 eq.) was added. The HCl salt precipitated rapidly. The suspension was stirred for 20 minutes at ambient temperature and the solid was collected by filtration. The filter cake was washed with THF (4 mL) and dried in a vacuum oven at 90° C. for 3 days to yield the title compound as a white solid which contained 0.4% CH₃CN.

m.p.=332-334° C.

MS: [M+H]⁺=395 (free base)

¹HNMR (500 MHz, DMSO-d₆): δ 7.10 (2H, br s), 7.39 (2H, t), 7.47 (2H, d), 7.57 (2H, d), 7.72 (1H, m), 8.00 (2H, br s), 9.84 ( s,1H ). Elemental Analysis for C₁₅H₁₃ClF₂N₆O₃S, MW=430.82:

Calculated: C, 41.82; H, 3.04; Cl, 8.23; F, 8.82; N, 19.51; S, 7.44.

Found: C, 42.04; H, 3.16; Cl, 8.13; F, 8.78, N, 19.50; S, 7.31.

### EXAMPLE 29

### HBr Salt of 4-[5-Amino-1-(2,6-difluoro-benzoyl)-1H-[1,2,4]triazol-3-ylamino]-benzenesulfonamide

A mixture of 4-[5-Amino-1-(2,6-difluoro-benzoyl)-1H-[1,2,4]triazol-3-ylamino]-benzenesulfonamide (2.0 gm) in THF (20 ml) was stirred at room temperature to effect a solution after which a 48% solution of aqueous HBr (0.56 ml, 0.95 eq.) was added. The HBr salt precipitated rapidly. The suspension was stirred for 20 minutes at ambient temperature and the solid was collected by filtration. The filter cake was washed with THF (4 mL) and dried in a vacuum oven at 90° C. for 3 days to yield the title compound as a white solid which contained 0.9% CH₃CN.m.p.=258-260° C.

MS: [M+H]⁺=395 (free base)

¹HNMR (500 MHz, DMSO-d₆): δ 7.20 (2H, br s), 7.39 (2H, t), 7.47 (2H, d), 7.58 (2H, d), 7.72, (1H, m), 8.01 (2H, br s), 9.84 (s, 1H). Elemental Analysis for for C₁₅H₁₃BrF₂N₆O₃S, MW=475.27:

Calculated: C, 37.91; H, 2.76; Br, 16.81; F, 7.99; N, 17.68; S, 6.75

Found: C, 38.10; H, 2.82; Br, 16.83; F, 7.76, N, 17.63; S, 6.72.

### EXAMPLE 30

### 0.5 H₂SO₄ Salt of 4-[5-Amino-1-(2,6-difluoro-benzoyl)-1H-[1,2,4]triazol-3-ylamino]-benzenesulfonamide

A mixture of 4-[5-Amino-1-(2,6-difluoro-benzoyl)-1H-[1,2,4]triazol-3-ylamino]-benzenesulfonamide (2.0 gm) in THF (40 ml) was stirred at room temperature to effect a solution after which, 96% H₂SO₄ (0.48 g, 0.95 eq.) was added. The—H₂SO₄ salt precipitated during 10 minutes. The suspension was stirred for an additional 20 minutes at ambient temperature and the solid was collected by filtration. The filter cake was washed with THF (4 mL) and dried in a vacuum oven at 90° C. for 3 days to yield the title compound as a white solid which contained 0.1% CH₃CN.

m.p.=293 -295° C.

MS: [M+H]⁺=395 (free base).

¹HNMR (500 MHz, DMSO-d₆): δ 7.09 (2H, br s), 7.39 (2H, t), 7.46 (2H, d), 7.57 (2H, d), 7.72, (1H, m), 8.00 (2H, br s), 9.84 (s, 1H). Elemental Analysis for C₁₅H₁₃F₂N₆O₅S_{1.5}, MW=443.40:

Calculated: C, 40.63; H, 2.96; F, 8.57; N, 18.95; S, 10.85

Found: C, 40.64; H, 2.90; F, 8.35; N, 18.79; S, 11.01.

### EXAMPLE 31

### N-[4-(aminosulfonyl)phenyl]-N′-cyanocarbamidic acid phenyl ester

A white slurry of diphenylcyanocarbonimidate (DPCCl) (810.31 g, 3.30 mol) in 12.0 L of dimethoxyethane (DME) was stirred and heated to 35° C. at which point all solids dissolved to yield a hazy solution. The solution was cooled to room temperature with precipitation of a small amount of DPCCl. A solution of 480 mL of 0.5 M ZnCl₂ in THF was added after which the reaction mixture was left to stir at room temperature. After stirring overnight the reaction mixture was cooled to 3° C. and 4-aminobenzenesulfonamide (600.0 g, 3.45 mol) was added. The resulting white suspension was stirred and heated to reflux (85° C.) as the solids dissolved. After about 1 h the product began to precipitate. The suspension was stirred at reflux for 7.5 h and then cooled to slowly to 0-5° C. The solid was collected by filtration, washed with 2.0 L of DME and dried in a vacuum oven overnight at 28″ Hg to yield N-[4-(aminosulfonyl)phenyl]-N′-cyanocarbamidic acid phenyl ester as a white solid. The material was used without further characterization in the next step.

HPLC purity: 95.7 wt %; 96.3 A %. KF: 0.34% H₂O

### EXAMPLE 32

### N³-[(4-aminosulfonyl)phenyl]-1-(2′,6′-difluorobenzoyl)-1H-1,2,4-triazole-3,5-diamine (Compound (Ia))

A mixture of N-[4-(aminosulfonyl)phenyl]-N′-cyanocarbamidic acid phenyl ester (1206.2 g, 3.60 mol), 2,6-difluorobenzoyl hydrazide (662.95 g, 3.85 mol) and pyridine (5.69 L) was stirred at about 20-30° C. until a solution was achieved. The reaction mixture was then heated to about 80-90° C. and held at this temperature for 6 h after which time the reaction was judged to be complete by HPLC analysis.

The yellow-brown solution was then cooled to about 20-30° C. and quenched into 7.5-8.0% aqueous NH₄Cl solution (30.2 L) while maintaining the quench solution at about 50-60° C. A solid was observed to precipitate during the quench. Methanol (1.00 L) was then added to the reaction mixture and the resulting off white suspension was stirred at 55-60° C. for 30 minutes and then cooled to 15-20° C. The solid was filtered, washed with water (4.55 L) and dried overnight in a vacuum oven at 90° C. to afford the crude product.

KF=2.5% H₂O

A suspension of the crude solid in 4.8 L of THF was heated to 55-60° C., stirred for 30 minutes, and then filtered to remove a small amount of insoluble material. The clear filtrate was distilled to remove about 2.8 L of THF after which 7.0 of acetonitrile was added and the slurry heated to 70° C. The resulting light tan slurry was cooled to 1.0° C. The suspension was filtered. After air drying overnight, the damp solid was suspended in 17.0 L of water, heated to about 100° C. and the suspension was distilled to remove about 4.0 L of solution. The slurry was cooled to 10-15° C. and the product was collected by filtration, washed with 2.0 L of water and dried in a vacuum oven at 90° C. and 28″ Hg to yield N³-[(4-aminosulfonyl)phenyl-1-(2′,6′-difluorobenzoyl)-1H-triazole-3,5-diamine as a white solid.

HPLC purity: 96.7 wt %; 99.0 A %. Elemental Analysis for C₁₅H₁₂F₂N₆O₃S×0.25 H₂O, MW=398.87:

Calculated: C, 45.17; H, 3.16; F, 9.53; N, 21.07; S, 8.04; H₂O, 1.13.

Found: C, 45.00; H, 2.97; F, 9.18; N, 20.94; S, 7.96; H₂O, 1.10.

Compounds #8, 13, 19, 24, 26, 27, 32, 33, 34, 36, 37 and 38 were similarly prepared according to the process of the present invention by reacting a suitably substituted hydrazide with a suitably N-substituted N′-cyano-carbamimidic acid phenyl ester under time and temperature conditions as listed in Table 5, below.

TABLE 5

| Compound # | Temp. | | Time |
|---|---|---|---|
| 8 | 85° | C. | 21.25 hr |
| 13 | 102-104° | C. | 5.5 hr |
| 19 | 102-104° | C. | 5.5 hr |
| 24 | 85° | C. | 7 hr |
| 26 | 85° | C. | 16 hr |
| 27 | 85° | C. | 16 hr |
| 32 | 105° | C. | 8 hr |
| 33 | 95-115° | C. | 24 hr |
| 34 | 95-115° | C. | 24 hr |
| 36 | 85-105° | C. | 8 hr |
| 37 | 85-105° | C. | 10 hr |

| | | |
|---|---|---|
| 38 | room temperature | 30 hr |

### EXAMPLE 33

### N³-[(4-aminosulfonyl)phenyl]-1-[3′-(trifluoromethyl)benzoyl]-1H-1,2,4-triazole-3,5-diamine (Compound #6)

To a clean, dry reaction tube was sequentially charged 3-(trifluoromethyl)benzoic hydrazide (0.94 g, 4.36 mmol), N-[4-(aminosulfonyl)phenyl]-N′-cyanocarbamidic acid phenyl ester (1.34 g, 4.15 mmol) and pyridine (10 mL). The suspension was stirred at room temperature for 5-10 min to effect solution after which time the reaction mixture was heated to 83° C. and stirred at 83-85° C. for 4 h. After 4 h the reaction mixture was cooled to room temperature and then added dropwise to of a vigorously stirred mixture of ice-H₂O (ca 200 mL). A fluffy, off-white solid precipitated. Solid sodium chloride (ca 20-25 gm) was added to the suspension which was stirred at 0-5° C. for 30 min and then filtered. The solid was washed with H₂O (ca 100 mL) and was air dried for 1 h. The damp solid was dried in a vacuum oven at 80° C. under a stream of nitrogen for 12 h to yield crude N³-[(4-aminosulfonyl)phenyl]-1-[3′-(trifluoromethyl )benzoyl]-1H-1,2,4-triazole-3,5-diamine as an off-white solid.

The crude product was dissolved in DMSO (4 mL) and purified on a silica gel column (30 g) using a mixture of ethyl acetate/n-heptane (70/30). The product containing fractions were combined and evaporated to yield an oily yellow solid containing residual DMSO, which was suspended in water (60 mL) and stirred at 50-55° C. for 30 min. The suspension was cooled to room temperature and filtered. The solid was then washed with water (30 mL). The product was dried in a vacuum oven at 80° C. for 16 h to yield N³-[(4-aminosulfonyl)phenyl]-1-[3′-(trifluoromethyl)benzoyl]-1H-1,2,4-triazole-3,5-diamine as a pale yellow solid.

HPLC purity: 98.5%

m.p. 251.0-253.0° C.

MS: [M+H]⁺=427, [M+Na]⁺=449

¹H NMR (300 MHz, DMSO-d₆): δ 7.15 (2H, s), 7.60-7.66 (4H, m), 7.84 (1H, t), 7.95 (2H, br s), 8.07 (1H, d), 8.33 (1H, d), 8.72 (1H, s), 9.87 (1H, s) Elemental Analysis for C₁₆H₁₃F₃N₆O₃S; MW=426.38

Calculated: C, 45.07; H, 3.07; N, 19.71; F, 13.37; S, 7.52

Found: C, 44.79; H, 2.94; N, 19.46; F, 12.92; S, 7.66.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of a compound of formula (I) 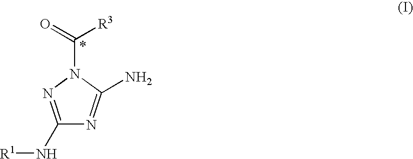
wherein
R₁ is selected from the group consisting of C₁₋₈alkyl, cycloalkyl, heterocyclyl, aryl and heteroaryl
wherein the cycloalkyl, heterocyclyl, aryl and heteroaryl are substituted with a substituent selected from the group consisting of:
(a) C₁₋₈alkyl (optionally substituted on a terminal carbon with a substituent selected from the group consisting of —C(O)H, —C(O)(C₁₋₈)alkyl, —CO₂(C₁₋₈)alkyl, amino, C₁₋₈alkylamino, di(C₁₋₈alkyl)amino, cyano, (halo)₁₋₃, hydroxy, nitro, cycloalkyl, heterocyclyl, aryl and heteroaryl),
(b) C₁₋₈alkoxy (optionally substituted on a terminal carbon with a substituent selected from the group consisting of (halo)₁₋₃ and hydroxy),
(c) —C(O)H, —C(O)(C₁₋₈)alkyl;
(d) —CO₂(C₁₋₈)alkyl;
(e) amino (substituted with two substituents independently selected from the group consisting of hydrogen, C₁₋₈alkyl and —SO₂—(C₁₋₈)alkyl),
(f) —C(O)amino (wherein amino is substituted with two substituents independently selected from the group consisting of hydrogen and C₁₋₈alkyl),
(g) —SO₂— {substituted with one substituent selected from the group consisting of heterocyclyl and amino (wherein amino is substituted with two substituents independently selected from the group consisting of hydrogen, C₁₋₈alkyl, —C₁₋₈alkylamino (wherein amino is substituted with two substituents independently selected from the group consisting of hydrogen and C₁₋₈alkyl) and heteroaryl)},
(h) cycloalkyl, heterocyclyl, aryl and heteroaryl
(wherein the cycloalkyl, heterocyclyl, aryl and heteroaryl are optionally substituted with 1 to 3 substituents independently selected from the group consisting of cyano, halo, hydroxy and nitro;
and wherein the heterocyclyl is optionally substituted with 1 to 2 oxo substituents; and, wherein cycloalkyl, heterocyclyl, aryl and heteroaryl are optionally substituted with a substituent selected from the group consisting of C₁₋₈alkyl (wherein alkyl is optionally substituted on a terminal carbon with a substituent selected from the group consisting of amino, C₁₋₈alkylamino, di(C₁₋₈alkyl)amino, cyano, (halo)₁₋₃, hydroxy and nitro), C₁₋₈alkoxy, amino, C₁₋₈alkylamino and di(C₁₋₈alkyl)amino);
R₃ is selected from the group consisting of: C₁₋₈alkyl, C₂₋₈alkenyl, C₂₋₈alkynyl
{wherein the C₁₋₈alkyl, C₂₋₈alkenyl and C₂₋₈alkynyl are optionally substituted on a terminal carbon with a substituent selected from the group consisting of —C(O)H, —C(O)(C₁₋₈)alkyl, —CO₂(C₁₋₈)alkyl, amino, C₁₋₈alkylamino, di(C₁₋₈alkyl)amino, cyano, (halo)₂₋₃, hydroxy, nitro, aryl and heteroaryl (wherein aryl and heteroaryl are optionally substituted with 1 to 5 substituents independently selected from the group consisting of C₁₋₈alkyl, cyano, (halo)₁₋₃(C₁₋₈)alkyl, (halo)₁₋₃(C₁₋₈)alkoxy, hydroxy, hydroxy(C₁₋₈)alkyl, hydroxy(C₁₋₈)alkoxy and nitro)},
cycloalkyl, heterocyclyl, aryl, heteroaryl
{wherein the cycloalkyl, heterocyclyl, aryl and heteroaryl are optionally substituted with 1 to 3 substituents independently selected from the group consisting of cyano, hydroxy and nitro;
wherein the aryl and heteroaryl are optionally substituted with (halo),₁₋₃;
and wherein the cycloalkyl, heterocyclyl, aryl and heteroaryl are optionally substituted with 1 to 2 substituents independently selected from the group consisting of:
(a) C₁₋₈alkyl, C₂₋₈alkenyl (wherein the C₁₋₈alkyl and C₂₋₈alkenyl are optionally substituted on a terminal carbon with a substituent selected from the group consisting of —C(O)H, —C(O)(C₁₋₈)alkyl, —CO₂(C₁₋₈)alkyl, amino, C₁₋₈alkylamino, di(C₁₋₈alkyl)amino, cyano, (halo)₂₋₃, hydroxy, nitro, cycloalkyl, heterocyclyl, aryl and heteroaryl),
(b) —CH(OH)—(C₁₋₈)alkyl,
(c) C₁₋₈alkoxy (optionally substituted on a terminal carbon with a substituent selected from the group consisting of (halo)₂₋₃ and hydroxy),
(d) —C(O)H, —C(O)(C₁₋₈)alkyl;
(e) —CO₂(C₁₋₈)alkyl;
(f) amino (substituted with two substituents independently selected from the group consisting of hydrogen, C₁₋₈alkyl and —C(O)(C₁₋₈)alkyl),
(g) —C(O)amino (wherein amino is substituted with two substituents independently selected from the group consisting of hydrogen and C₁₋₈alkyl),
(h) —SO₂— {substituted with one substituent selected from the group consisting of heterocyclyl and amino (wherein amino is substituted with two substituents independently selected from the group consisting of hydrogen, C₁₋₈alkyl and —C₁₋₈alkylamino (wherein amino is substituted with two substituents independently selected from the group consisting of hydrogen and C₁₋₈alkyl))},
(i) —NH—SO₂—(C₁₋₈)alkyl,
(j) cycloalkyl, heterocyclyl (optionally substituted with 1 to 2 oxo substituents), aryl and heteroaryl} and
amino;
wherein the amino group is substituted with two substituents independently selected from the group consisting of hydrogen, C₁₋₈alkyl, cycloalkyl, aryl and heteroaryl (wherein the cycloalkyl, aryl and heteroaryl are optionally substituted with 1 to 5 substituents independently selected from the group consisting of C₁₋₈alkyl, cyano, (halo)₁₋₃(C₁₋₈)alkyl, (halo)₁₋₃(C₁₋₈)alkoxy, hydroxy, hydroxy(C₁₋₈)alkyl, hydroxy(C₁₋₈)alkoxy and nitro);
provided that when R³ is aryl or heteroaryl, wherein the aryl or heteroaryl is optionally substituted with a —(CH₂)₀₋₂—CO₂(C₁₋₈)alkyl group, then the —(CH₂)₀₋₂—CO₂(C₁₋₈)alkyl group is not bound at the ortho position relative to the bond identified by the asterisk in the compound of formula (I);
provided further that when R³ is cycloalkyl or a heterocyclyl, wherein the cycloalkyl or heterocyclyl is optionally substituted, then the substituent on the cycloalkyl or heterocyclyl is other than —(CH₂)₀₋₂—CO₂(C₁₋₈)alkyl;
and pharmaceutically acceptable salts thereof;
comprising 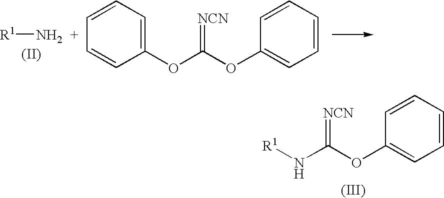
reacting a suitably substituted compound of formula (II) with diphenyl cyanocarbonimidate, in a first organic solvent, to yield the corresponding compound of formula (III); 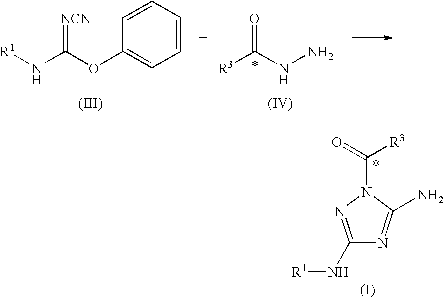
reacting the compound of formula (III) with a suitably substituted compound of formula (IV), in a second organic solvent, to yield the corresponding compound of formula (I).

2. A process as in claim 1, wherein the first organic solvent is pyridine.

3. A process as in claim 2, wherein the second organic solvent is pyridine.

4. A process as in claim 1, wherein the compound of formula (II) is reacted with diphenyl cyanocarbonimidate in the presence of a Lewis acid catalysts or a first inorganic or organic base.

5. A process as in claim 4, wherein the compound of formula (II) is reacted with diphenyl cyanocarbonimidate in the presence of a first organic base.

6. A process as in claim 5, wherein the first organic base is a tertiary amine base.

7. A process as in claim 6, wherein the tertiary amine base is pyridine.

8. A process as in claim 1, wherein the compound of formula (III) is reacted with the compound of formula (IV) in the presence of a second inorganic or organic base.

9. A process as in claim 8, wherein the compound of formula (III) is reacted with the compound of formula (IV) in the presence of a second organic base.

10. A process as in claim 9, wherein the second organic base is a tertiary amine base.

11. A process as in claim 10, wherein the tertiary amine base is pyridine.

12. A process as in claim 1, wherein the compound of formula (III) is reacted with the compound of formula (IV) at a temperature in the range of about 80 to about 120° C.

13. A process as in claim 12, wherein the compound of formula (III) is reacted with the compound of formula (IV) at a temperature in the range of about 80 to about 90° C.

14. A process as in claim 1, wherein R¹ is 4-aminosulfonylphenyl and wherein R³ is 2,6-difluorophenyl.

15. A process for the preparation of a compound of formula (Ia) 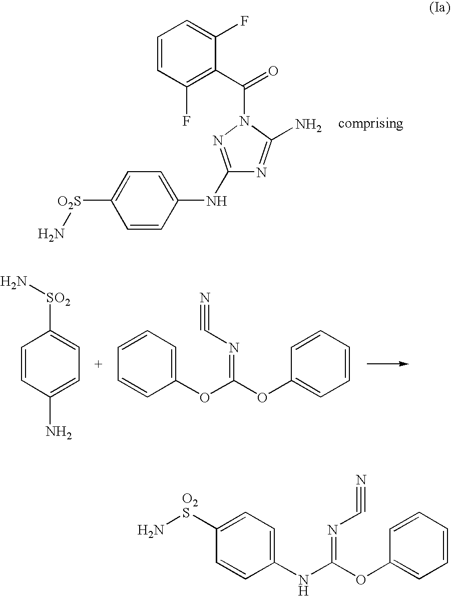
reacting 4-aminobenzenesulfonamide with diphenyl cyanocarbonimidate, in a first organic solvent, to yield N-[4-(aminosulfonyl)phenyl]-N′-cyanocarbamidic acid phenyl ester; 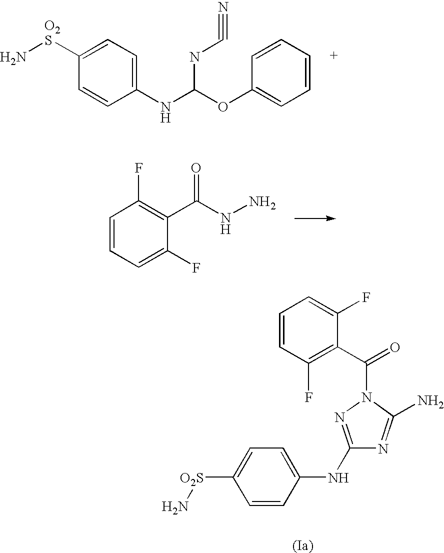
reacting N-[4-(aminosulfonyl)phenyl]-N′-cyanocarbamidic acid phenyl ester with 2,6-difluorobenzoic acid hydrazide, in a second organic solvent, to yield the corresponding compound of formula (Ia).

16. A process as in claim 15, wherein the first organic solvent is pyridine.

17. A process as in claim 16, wherein the second organic solvent is pyridine.

18. A process as in claim 15, wherein the compound of formula (II) is reacted with diphenyl cyanocarbonimidate in the presence of a Lewis acid catalysts or a first inorganic or organic base.

19. A process as in claim 18, wherein the compound of formula (II) is reacted with diphenyl cyanocarbonimidate in the presence of a first organic base.

20. A process as in claim 19, wherein the first organic base is a tertiary amine base.

21. A process as in claim 20, wherein the tertiary amine base is pyridine.

22. A process as in claim 15, wherein the compound of formula (III) is reacted with the compound of formula (IV) in the presence of a second inorganic or organic base.

23. A process as in claim 22, wherein the compound of formula (III) is reacted with the compound of formula (IV) in the presence of a second organic base.

24. A process as in claim 23, wherein the second organic base is a tertiary amine base.

25. A process as in claim 24, wherein the tertiary amine base is pyridine.

26. A process as in claim 15, wherein the compound of formula (III) is reacted with the compound of formula (IV) at a temperature in the range of about 80 to about 120° C.

27. A process as in claim 26, wherein the compound of formula (III) is reacted with the compound of formula (IV) at a temperature in the range of about 80 to about 90° C.

28. A compound prepared according to the process as in claim 1.

29. A pharmaceutical composition comprising a pharmaceutically acceptable carrier and a compound of claim 28.

30. A pharmaceutical composition made by mixing a compound of claim 28 and a pharmaceutically acceptable carrier.

31. A process for making a pharmaceutical composition comprising mixing a compound of claim 28 and a pharmaceutically acceptable carrier.

32. A method of treating or ameliorating a kinase or dual-kinase mediated disorder, in a subject in need thereof comprising administering to the subject a therapeutically effective amount of the compound of claim 28.

33. A compound prepared according to the process as in claim 15.

34. A pharmaceutical composition comprising a pharmaceutically acceptable carrier and a compound of claim 33.

35. A pharmaceutical composition made by mixing a compound of claim 33 and a pharmaceutically acceptable carrier.

36. A process for making a pharmaceutical composition comprising mixing a compound of claim 33 and a pharmaceutically acceptable carrier.

37. A method of treating or ameliorating a kinase or dual-kinase mediated disorder, in a subject in need thereof comprising administering to the subject a therapeutically effective amount of the compound of claim 33.

38. A crystalline form of 4-[5-Amino-1-(2,6-difluoro-benzoyl)-1H-[1,2,4]triazol-3-ylamino]-benzenesulfonamide comprising the following X-ray diffraction pattern

| Pos. [°2Theta] | Rel. Int. [%] |
|---|---|
| 5.21 | 21.24 |
| 10.39 | 14.40 |
| 13.71 | 29.54 |
| 15.58 | 87.39 |
| 17.00 | 25.38 |
| 17.20 | 27.26 |
| 18.02 | 40.96 |
| 18.71 | 23.97 |
| 19.24 | 39.50 |
| 19.63 | 54.58 |
| 20.11 | 38.33 |
| 21.27 | 45.19 |
| 21.43 | 47.58 |
| 22.69 | 15.18 |
| 23.20 | 91.38 |
| 23.82 | 100.00 |
| 24.91 | 13.59 |
| 26.08 | 35.19 |
| 27.56 | 57.62 |
| 27.78 | 55.67 |
| 28.19 | 53.70 |
| 30.09 | 14.96 |
| 32.22 | 11.43 |
| 32.45 | 11.52 |

39. A crystalline form of 4-[5-Amino-1-(2,6-difluoro-benzoyl)-1H-1,2,4]triazol-3-ylamino]-benzenesulfonamide characterized by a melt endotherm with a peak temperature at about 242° C.

40. A process as in claim 15, wherein the 4-aminobenzenesulfonamide is reacted with diphenyl cyanocarbonimidate in the absence of a catalyst; and wherein the N-[4-(aminosulfonyl)phenyl]-N′-cyanocarnamidic acid ester is not isolated prior to reacting the N-[4-(aminosulfonyl)phenyl]-N′-cyanocarnamidic acid ester with 2,6-difluorobenzoic acid hydrazide.

41. A process for the preparation of the crystalline form of 4-[5-Amino-1-(2,6-difluoro-benzoyl)-1H-[1,2,4]triazol-3-ylamino]-benzenesulfonamide as in claim 38 comprising
(a) dissolving a mixture of crystalline forms of 4-[5-Amino-1-(2,6-difluoro-benzoyl)-1H-[1,2,4]triazol-3-ylamino]-benzenesulfonamide in an organic solvent;
(b) reacting the mixture of step (a) with hydrochloric acid to yield the HCl salt of 4-[5-Amino-1-(2,6-difluoro-benzoyl)-1H-[1,2,4]triazol-3-ylamino]-benzenesulfonamide;
(c) isolating the HCl salt of 4-[5-Amino-1-(2,6-difluoro-benzoyl)-1H-[1,2,4]triazol-3-ylamino]-benzenesulfonamide;
(d) suspending the HCl salt of 4-[5-Amino-1-(2,6-difluoro-benzoyl)-1H-[1,2,4]triazol-3-ylamino]-benzenesulfonamide in water and stirring to a constant pH.

42. A product prepared according to the process as in claim 40.

43. A pharmaceutical composition comprising a pharmaceutically acceptable 25 carrier and a compound of claim 42.

44. A pharmaceutical composition made by mixing a compound of claim 43 and a pharmaceutically acceptable carrier.

45. A process for making a pharmaceutical composition comprising mixing a compound of claim 42 and a pharmaceutically acceptable carrier.

46. A method of treating or ameliorating a kinase or dual-kinase mediated disorder, in a subject in need thereof comprising administering to the subject a therapeutically effective amount of the compound of claim 42.

47. A crystalline form of 4-[5-Amino-1-(2,6-difluoro-benzoyl)-1H-[1,2,4]triazol-3-ylamino]-benzenesulfonamide comprising the following X-ray diffraction pattern

| Pos. [°2Theta] | Rel. Int. [%] |
|---|---|
| 12.87 | 10.11 |
| 13.74 | 17.65 |
| 14.74 | 100.00 |
| 15.26 | 21.35 |
| 15.44 | 12.37 |
| 18.15 | 23.77 |
| 19.45 | 28.96 |
| 19.67 | 28.55 |
| 20.29 | 15.34 |
| 20.55 | 15.89 |
| 20.77 | 11.87 |
| 21.27 | 16.03 |
| 21.47 | 11.42 |
| 22.06 | 10.74 |
| 24.69 | 40.20 |
| 25.46 | 12.51 |
| 25.78 | 14.85 |
| 26.21 | 17.42 |
| 26.72 | 24.18 |
| 27.17 | 15.01 |
| 28.50 | 17.31 |
| 28.78 | 25.55 |

48. A process as in claim 15, wherein the 4-aminobenzenesulfonamide is reacted with diphenyl cyanocarbonimidate in the presence of ZnCl₂; and wherein the N-[4-(aminosulfonyl)phenyl]-N′-cyanocarnamidic acid ester is isolated prior to reacting the N-[4-(aminosulfonyl)phenyl]-N′-cyanocarnamidic acid ester with 2,6-difluorobenzoic acid hydrazide.

49. A product prepared according to the process as in claim 48.

50. A CH₃SO₃H salt of 4-[5-Amino-1-(2,6-difluoro-benzoyl)-1H-[1,2,4]triazol-3-ylamino]-benzenesulfonamide.

51. A CH₃SO₃H salt as in claim 50, wherein the molar ratio of 4-[5-Amino-1-(2,6-difluoro-benzoyl)-1H-[1,2,4]triazol-3-ylamino]-benzenesulfonamide to CH₃SO₃H is 1:1.

52. A CH₃SO₃H salt as in claim 50, comprising the following X-ray diffraction pattern

| Pos. [°2Theta] | Rel. Int. [%] |
|---|---|
| 15.89 | 62.06 |
| 17.43 | 27.06 |
| 18.76 | 25.76 |
| 19.88 | 46.91 |
| 20.26 | 40.61 |
| 20.92 | 51.81 |
| 21.44 | 87.25 |
| 22.18 | 72.66 |
| 22.76 | 59.56 |
| 26.51 | 32.29 |
| 27.08 | 100.00 |
| 28.59 | 12.36 |
| 33.36 | 11.20 |

53. A process for the preparation of a CH₃SO₃H salt of 4-[5-Amino-1-(2,6-difluoro-benzoyl)-1H-[1,2,4]triazol-3-ylamino]-benzenesulfonamide comprising reacting 4-[5-Amino-1-(2,6-difluoro-benzoyl)-1H-[1,2,4]triazol-3-ylamino]-benzenesulfonamide with CH₃SO₃H.

54. A HCl salt of 4-[5-Amino-1-(2,6-difluoro-benzoyl)-1H-[1,2,4]triazol-3-ylamino]-benzenesulfonamide.

55. A HCl salt as in claim 54, wherein the molar ratio of 4-[5-Amino-1-(2,6-difluoro-benzoyl)-1H-[1,2,4]triazol-3-ylamino]-benzenesulfonamide to HCl is 1:1.

56. A HCl salt as in claim 54, comprising the following X-ray diffraction pattern

| Pos. [°2Theta] | Rel. Int. [%] |
|---|---|
| 13.67 | 45.30 |
| 14.27 | 43.44 |
| 15.85 | 33.11 |
| 17.01 | 45.04 |
| 17.18 | 52.13 |
| 17.54 | 40.78 |
| 18.21 | 31.62 |
| 19.36 | 63.78 |
| 20.36 | 43.04 |
| 21.20 | 32.54 |
| 22.45 | 40.97 |
| 22.98 | 65.31 |
| 23.75 | 100.00 |
| 25.36 | 21.59 |
| 26.09 | 13.23 |
| 26.82 | 40.99 |
| 27.23 | 77.86 |
| 27.70 | 74.23 |
| 28.73 | 12.94 |
| 34.04 | 16.93 |

57. A process for the preparation of a HCl salt of 4-[5-Amino-1-(2,6-difluoro-benzoyl)-1H-[1,2,4]triazol-3-ylamino]-benzenesulfonamide comprising reacting 4-[5-Amino-1-(2,6-difluoro-benzoyl)-1H-[1,2,4]triazol-3-ylamino]-benzenesulfonamide with HCl.

58. A HBr salt of 4-[5-Amino-1-(2,6-difluoro-benzoyl)-1H-[1,2,4]triazol-3-ylamino]-benzenesulfonamide.

59. A HBr salt as in claim 58, wherein the molar ratio of 4-[5-Amino-1-(2,6-difluoro-benzoyl)-1H-[1,2,4]triazol-3-ylamino]-benzenesulfonamide to HBr is 1:1.

60. A HBr salt as in claim 58, comprising the following X-ray diffraction pattern

| Pos. [°2Theta] | Rel. Int. [%] |
|---|---|
| 4.46 | 47.43 |
| 13.40 | 17.79 |
| 15.75 | 33.50 |
| 16.99 | 33.36 |
| 17.40 | 77.64 |
| 17.99 | 30.47 |
| 19.31 | 45.07 |
| 20.31 | 45.66 |
| 20.63 | 44.81 |
| 21.13 | 47.54 |
| 22.19 | 32.71 |
| 22.47 | 39.15 |
| 22.68 | 27.02 |
| 23.81 | 83.64 |
| 23.99 | 79.30 |
| 25.10 | 48.15 |
| 26.01 | 13.57 |
| 27.35 | 100.00 |
| 28.03 | 21.98 |
| 31.60 | 25.34 |
| 33.57 | 31.35 |

61. A process for the preparation of a HBr salt of 4-[5-Amino-1-(2,6-difluoro-benzoyl)-1H-[1,2,4]triazol-3-ylamino]-benzenesulfonamide comprising reacting 4-[5-Amino-1-(2,6-difluoro-benzoyl )-1H-[1,2,4]triazol-3-ylamino]-benzenesulfonamide with HBr.

62. A H₂SO₄ salt of 4-[5-Amino-1-(2,6-difluoro-benzoyl)-1H-[1,2,4]triazol-3-ylamino]-benzenesulfonamide.

63. A H₂SO₄ salt as in claim 62, wherein the molar ratio of 4-[5-Amino-1-(2,6-difluoro-benzoyl)-1H-[1,2,4]triazol-3-ylamino]-benzenesulfonamide to H₂SO₃₄ is 1:0.5.

64. A H₂SO₄ salt as in claim 62, comprising the following X-ray diffraction pattern

| Pos. [°2Theta] | Rel. Int. [%] |
|---|---|
| 4.68 | 72.76 |
| 7.63 | 42.65 |
| 9.37 | 15.29 |
| 13.06 | 55.75 |
| 13.51 | 87.87 |
| 14.38 | 24.75 |
| 14.98 | 74.53 |
| 15.29 | 100.00 |
| 15.84 | 18.68 |
| 16.44 | 21.95 |
| 16.80 | 37.42 |
| 17.34 | 17.66 |
| 17.62 | 25.79 |
| 18.40 | 62.45 |
| 18.81 | 68.51 |
| 19.53 | 67.69 |
| 19.60 | 60.93 |
| 20.04 | 91.72 |
| 20.29 | 94.30 |
| 21.28 | 49.73 |
| 22.62 | 54.35 |
| 23.03 | 80.37 |
| 23.78 | 28.94 |
| 24.49 | 84.20 |
| 25.22 | 41.07 |
| 25.63 | 67.44 |
| 26.62 | 61.21 |
| 27.88 | 23.65 |
| 28.40 | 36.08 |
| 29.38 | 14.51 |
| 30.91 | 24.95 |
| 32.11 | 28.93 |
| 33.02 | 14.66 |
| 33.42 | 18.68 |

65. A process for the preparation of a H₂SO₄ salt of 4-[5-Amino-1-(2,6-difluoro-benzoyl)-1H-[1,2,4]triazol-3-ylamino]-benzenesulfonamide comprising reacting 4-[5-Amino-1-(2,6-difluoro-benzoyl)-1H-[1,2,4]triazol-3-ylamino]-benzenesulfonamide with H₂SO₄.
